(19) United States
(12) Patent Application Publication
(10) Pub. No.: US 2025/0210175 A1
(43) Pub. Date: Jun. 26, 2025

(54) COMPUTER SYSTEM AND METHOD FOR CALCIUM CITRATE HEMOFILTER DECISION SUPPORT

(71) Applicant: Universität Zürich, Zürich (CH)

(72) Inventors: Justyna Lunkiewicz, Mellingen (CH); David W. Tscholl, Zürich (CH)

(21) Appl. No.: 18/393,027

(22) Filed: Dec. 21, 2023

(51) Int. Cl. G16H 20/17; A61B 5/026; A61B 5/145

(52) U.S. Cl. CPC G16H 20/17; A61B 5/026; A61B 5/14542

(57) ABSTRACT

To monitor a clinical state of a patient during calcium citrate hemofilter therapy, blood gas analysis sensor data is received as time series of sampled measurement values for the patient's blood gas state parameters, and, time series of continuous measurement values for calcium citrate hemofilter state parameters are received. Each blood gas analysis state parameter is mapped to predefined calcium citrate hemofilter adjustment reference tables defining whether a parameter is within a normal range, and generating one or more therapy adjustment suggestion outputs based on one or more of the blood gas analysis state parameters. Each state parameter is mapped to a predefined corresponding graphical representation. A virtual scene representation is rendered representing the inside of a blood filled hemofilter catheter representing blood flowing through the hemofilter. Thereby, animated visualizations of the graphical representations reflect current values of the respective state parameters and the generated therapy adjustment suggestion outputs.

### TECHNICAL FIELD

The present description generally relates to the field of monitoring the clinical state of a patient. In particular, the description relates to methods, computer program products and systems for supporting a respectively trained person in calcium citrate hemofilter therapy of a patient.

### BACKGROUND

Calcium citrate hemofilter (CaCi hemofilter) is a recommended form of continuous renal replacement therapy (CRRT) for hemodynamically unstable patients with severe acute kidney injury (AKI). Some of the metabolic complications related to calcium citrate hemofilter therapy may be prevented by a prompt therapy adjustment. The maintenance of the CaCi hemofilter, the monitoring of blood gas analysis and the necessary adjustments of the parameter settings are time-consuming. They require several steps and a high level of concentration by a respectively trained medical person. Blood gas tests are typically displayed in numerical values in a hospital information system. A blood gas analysis monitoring system may display fifty or more different raw data points on a single screen. However, the number of displayed data points is huge and overwhelms users with information, especially if only a few data points are required for monitoring of calcium citrate hemofilter and therapy adjustments. With a growing number of monitored parameters, it becomes impossible for the human visual system to process all the observed information such that the human brain can consistently comprehend the monitored data and remain aware of the patient's situation (i.e., the patient's medical/clinical state) and calcium citrate hemofilter paraments requiring adjustment. However, a correct and rapid understanding of a patient's blood test results by the attending medically trained person (e.g., a physician) is vital for patient care. Only when the blood gas diagnostic data is correctly understood and required adjustments to calcium citrate hemofilter can be identified from such data, and when there is a high degree of certainty with regard to the understanding and relevance of the data, can a physician quickly initiate further diagnostic steps or prescribe the appropriate therapy. Such decisions typically need to be taken in real-time to avoid that the patient passes into a critical or even life-threatening state.

### SUMMARY

Hence, there is a need for system and method to provide a diagnostic and decision support tool to medically trained users which allow such users a quick and intuitive assessment of the patient's overall medical state during calcium citrate hemofilter therapy and required therapeutic adjustment with a high level of confidence. This enables such users to quickly arrive at correct diagnoses and apply appropriate therapeutic treatment to the patient if required.

The herein disclosed approach allows such monitoring of the clinical state of a patient during calcium citrate hemofilter therapy to support the selection of required adjustments to the calcium citrate hemofilter therapy (by a medically trained person) as needed by an appropriate synthesis of blood test system monitoring information, current calcium citrate hemofilter state parameter settings and calcium citrate hemofilter reference tables for therapy adjustment for a user of a calcium citrate hemofilter device. Such a user can also be a personal consumer using an e-health app and having an adequate level of medical training. In particular, rendering of appropriate representations of blood gas state parameters of the patient and calcium citrate hemofilter state parameters allows the human visual system of such users to extract relevant information from the monitored data with high accuracy and take time-critical reliable therapeutic decisions. Thereby, it is to be noted that a correct and rapid assessment of a patient's blood test results by the attending medically trained person is vital for patient care. It has been shown in respective studies that clear visualizations of the required change to calcium citrate hemofilter settings optimized for recognition by the human visual system are a key element of a solution solving the above technical problem.

In one embodiment, the above technical problem is solved by a computer-generated medical instrument, comprising a graphical display, synthesizing raw blood test function data (arterial blood gas state and postfilter blood gas parameters) and therapy adjustments recommendations according to the reference adjustments tables for calcium citrate hemofilter into a single graphical two- or three-dimensional synthetic blood vessel model. The blood vessel model corresponds to the inside of an artificial catheter filled with blood through which an animated flow of graphical representation objects of relevant blood components can be observed. Such representation objects represent the condition of the monitored patient's blood test results and calcium citrate hemofilter state parameters settings (also referred to as citrate hemofilter state parameters) according to the raw data input. In case of required adjustments to the calcium citrate hemofilter state parameter settings, such adjustments are also represented by respective graphical representation objects. Compared to a conventional monitoring device, the herein disclosed computer-generated user interface is much more easily perceived and assessed by users with a respective level of medical training. This can be particularly advantageous when the user must make quick decisions under stress.

In other words, with this computer-generated medical instrument, the relevant patient information is better perceived by the human visual system and the human neurocognitive system and thus the information is better processed than when users use conventional presentation of laboratory values as provided by prior art tools. This benefit of improved perception through the human visual/neurocognitive systems was demonstrated in a study with 26 participants which is described in detail in the detailed description part of this document. Participating users were more likely to make the correct therapeutic decisions, perceived less cognitive workload and had greater decision-making confidence when they used the herein disclosed computer-generated instrument rather than the corresponding conventional presentation of blood gas analysis results and standard reference tables. The computer-generated medical instrument is adapted to animate various graphical objects such that they move at a certain pace and interact with each other intuitively representing the interaction of arterial, venous and postfilter blood gas state parameters derived from a blood gas analysis machine in reference to standard reference therapy adjustment tables and their meaning in case therapy adjustment is needed. By using such graphical animated representations of the patient's blood gas parameters (from arterial or venous and postfilter blood gas analysis) relevant to calcium citrate hemofilter therapy, relating them to the reference adjustment tables and visually displaying required adjustments to be made, a human user perceives the relevant blood gas state parameter information of the patient directly from such representations. From a corresponding study it is known that the human visual system supports the user to retrieve such information without a need for mentally translating parameter values (numbers) all the time, analyzing them according to respective tables and identifying required adjustments required to be made to maintain adequate calcium citrate hemofilter therapy.

In the following, various embodiments of the herein disclosed approach are described. Such embodiments comprise a computer-implemented method, a computer readable medium (computer program product) comprising program instructions that, when loaded into a memory of a computing device and executed by at least one processor of the computing device, cause the at least one processor to execute said method, and a computer system which is adapted to execute said method when executing said computer program.

In a first aspect, a computer-implemented method is provided for rendering and displaying representations of calcium citrate hemofilter therapy relevant blood gas state parameters of a patient and calcium citrate hemofilter state parameters to support a medically trained person in monitoring and adjusting calcium citrate hemofilter therapy of the patient. The method comprises:
receiving, from a data source, time series of sampled measurement values obtained from a plurality of blood gas analysis (arterial/venous and postfilter blood gas analysis) sensors for the following blood gas state parameters of the patient: systemic ionized calcium (arterial or venous blood gas), postfilter calcium (post filter blood gas), pH (arterial blood gas), bicarbonate (arterial blood gas), sodium, potassium and chloride (arterial or venous blood gas);
mapping each state parameter to standard reference adjustments tables defining if a parameter is within a normal range, too low or too high and what adjustments are required to return all parameters to normal range;
mapping each state parameter output to a predefined corresponding graphical representation with each graphical representation for a particular state parameter being distinct from all graphical representations of the remaining state parameters. For example, elliptic objects and/or droplet objects may be used for the representation of respective different state parameter types such that the rendered representation clearly distinguish. E.g., the ellipses used for the different parameter types may distinguish through object properties such as the color of the object, the size and/or shape of the object (e.g., the sizes of main and secondary axes of the ellipses), object labels, etc.; and
rendering, in a virtual scene representation of the inside of a blood filled hemofilter catheter representing blood flowing through the inside of an hemofilter catheter, animated visualizations of the graphical representations, in accordance with predefined animation rules, such that respective graphical objects move through the filter inside the blood hemofilter catheter to illustrate the parameters flow and reflect current values of the respective state parameters and any adjustments required to be made.

The rendered animated scene is then displayed on the graphical interface of the computer-generated medical instrument. Thereby, in a basic embodiment, the predefined animation rules may comprise:
For the calcium citrate hemofilter settings parameters, such as blood flow and dialysate flow, citrate infusion rate, calcium infusion rate:
Flow rate of red blood cells (showing blood flow), dialysate droplets (showing dialysate flow), calcium ions (showing calcium infusion flow) and calcium ions (showing calcium infusion rate) at a standard animation pace approx. 8-12 mm/s indicating adequate (normal) flow setting for the patient's current state,
Flow rate of red blood cells (showing blood flow), dialysate droplets (showing dialysate flow), calcium ions (showing calcium infusion flow) and calcium ions (showing calcium infusion rate) at two to four times slower pace (and automatically two to four times lower number) than the standard animation pace, indicating a too low rate for the patient's state suggesting it's increase,

Flow rate of red blood cells (showing blood flow), dialysate droplets (showing dialysate flow), calcium ions infused after the filter (showing calcium infusion flow) and citrate ions (showing citrate infusion rate) at two to four times higher (faster) pace (automatically at two to four times higher times greater number) than the standard animation pace indicating a too high rate of the parameter for the patient's state, hence requiring reduction.

For the calcium citrate hemofilter state parameters such as citrate infusion, calcium infusion and dialysate flow further predefined animation rules comprise:
If the infusion rate of citrate, calcium and dialysate is within normal limits and does not require rate changing, there is an infusion source object having a predefined standard size representing an infusion bag of a solution,
If the infusion rate of citrate, calcium and dialysate is too high and should be reduced, the size of the infusion source object (i.e., the object representing the source of infused solution) is enlarged by at least factor 1.5 compared to the predefined standard size representing this parameter within a normal range. The contour of the visualization of the infusion source object may be shown as a thickened-line.
If the infusion rate of citrate, calcium and dialysate is too low and should be increased, the size of the infusion source object is reduced by at least factor 1.5 compared to the predefined standard size. The contour of the visualization of the infusion source object may be rendered as a thin dashed-line,

For the calcium citrate hemofilter state parameters such as citrate infusion, calcium infusion further predefined animation rules comprise:
If the individual infusion rate of citrate or calcium is within normal limits and does not require rate changing, individual citrate and calcium objects may have a continuous contour.
If the individual infusion rate of citrate or calcium is too high and require rate reduction, individual citrate and calcium objects may have a continuous and/or thickened contour.
If the individual infusion rate of citrate or calcium is too low and should be increased, the visualization of the individual ions may have a dashed-line contour.

For the systemic calcium state parameter:
Systemic calcium ions (measured in arterial or venous blood gas analysis) are represented by systemic calcium ion objects flowing from the left-hand side towards the filter (before filter) with a 1:1 ratio to citrate ion objects representing the infused citrate ions indicating normal systemic calcium concentration.
Systemic calcium ion objects flowing from the left-hand side towards the filter (before filter) in a systemic calcium to infused citrate ratio ranging from 1:2 to 1:5 (ratio used in a study 1:3), indicate too low systemic calcium concentration,
Systemic calcium ion objects flowing from the left-hand side towards the filter (before filter) in a systemic calcium to infused citrate ratio ranging from 5:1 to 2:1 (ratio used in a study 3:1), indicate too high systemic calcium concentration,

For the systemic calcium state parameter:
If systemic calcium ions (measured in arterial or venous blood gas analysis) flowing from the left-hand side towards the filter (before filter) are within normal limits, individual systemic calcium ion objects have a continuous line contour, and flow at a single level in a stream of objects of a blood-filled catheter. In other words, animated (flowing) objects form layers in the 2D view of the vessel and systemic calcium flows in this case in one layer only, i.e., at a single level).
If systemic calcium ions (measured in arterial or venous blood gas analysis) flowing from the left-hand side towards the filter (before filter) exceed limits (are too high), individual systemic calcium ion objects have a continuous line contour and flow at multiple levels in a stream of objects through the blood-filled catheter. In other words, the animated objects form layers and systemic calcium flows, in this case, in multiple layers, i.e., at multiple levels).
If systemic calcium ions (measured in arterial or venous blood gas analysis) flowing from the left-hand side towards the filter (before filter) are too low, individual systemic calcium ion objects have a dashed-line contour and flow in a single level of the blood-filled catheter.

For the postfilter calcium concentration state parameter:
If a postfilter calcium level is within normal limits, there are no extra representation objects drawing attention towards the postfilter calcium concentration measured in postfilter calcium blood gas analysis,
If a postfilter calcium level is measured as too low in postfilter calcium blood gas analysis, this is indicated by a single blinking (periodically disappearing) calcium ion object in the “post filter” part of the blood-filled catheter which, in addition, may have a dashed line contour.
If a postfilter calcium level is measured as too high in a postfilter calcium blood gas analysis, this is indicated by a cluster of a plurality of multiple blinking calcium ion objects (e.g., six extra calcium ion objects) in the “post filter” part of the blood-filled catheter.

For the metabolic alkalosis or acidosis state parameters:
A 1:1 ratio of hydrogen (H+) to bicarbonate ions (HCO₃⁻) objects indicates normal pH of a patient (neither metabolic alkalosis or acidosis state) measured in arterial blood gas analysis of the patient.
A ratio of hydrogen (H+) to bicarbonate ion (HCO₃⁻) objects in the range from 1:2 to 1:5 (advantageously 1:3) indicates metabolic alkalosis state, where hydrogen ions (H+) objects may have a dashed line contour, a 3:1 ratio of hydrogen (H+) to bicarbonate ions (HCO₃⁻) objects, in the range from 2:1 to 5:1 (advantageously 3:1) indicates metabolic acidosis state, where bicarbonate ions (labelled HCO₃⁻) objects may have a dashed line contour.

For the citrate accumulation state parameters:
in case of no-citrate accumulation state, i.e., a normal state anion gap symbol (A−) having a predefined normal size in comparison to other flowing objects is present within a plurality of previously specified number of objects in the range of 1 to 5 (two objects were used in the study) in a 10 to 50 seconds long stream visualization (25 seconds were used in the study). Thereby, the systemic calcium concentration depends on the actual measured systemic calcium concentration (either too low, too high or within normal limits). To diagnose citrate accumulation, the measurement of systemic calcium concentration is required. If there is no citrate accumulation, visualization of systemic calcium concentration is either low, normal or high, depending on what the patient's systemic calcium concentration actually is.
If, however, there is citrate accumulation, then the anion gap is big and systemic calcium concentration is low. That is, in case of citrate accumulation: the anion gap symbol is increased in size compared to the normal size (e.g., doubled) and low systemic calcium concentration is visualized as described above.

Thereby, the anion gap may be calculated according any of the following equations:

anion gap=([Na⁺]+[K⁺])−([Cl⁻]+[HCO₃⁻] (E1), or

anion gap=[Na⁺]−([Cl⁻]+[HCO₃⁻]) (E2),
where Na+ (sodium ions), K+ (potassium ions), Cl⁻ (Chloride ions), HCO₃⁻ ions (bicarbonate ions) concentrations are extracted from a blood gas analysis for the respective patient.

It is to be noted that the patient's blood constituents objects move through the inside of the blood-filled catheter for hemofiltration representation (i.e., the scene) at the same speed as their associated red blood cell objects indicating blood flow with the predefined animation rules.

In one embodiment, objects in the scene may have two alternating states comprising a solid object visualization state and a non-solid object state. An object in the non-solid object state indicating too low concentration or too low rates requiring an adjustment may be outlined with dashed lines. In addition, in the non-solid object state the shape of the object may originate from an infusion source object that is at least a factor 1.5 smaller in size compared to the predefined standard size. This reduced object may also be outlined with dashed lines.

An object in the non-solid object state indicating too high concentration or too high rates requiring an adjustment may be outlined with thickened lines. In addition, in the non-solid object state, the shape of the object may originate from an infusion source object that is at least a factor 1.5 bigger in size compared to the predefined standard size.

In one embodiment, the method may further comprise:
receiving, from the data source, further time series of sampled measurement values for a postfilter calcium concentration state parameter of the patient (postfilter blood gas analysis);
mapping the postfilter calcium concentration state parameter to a predefined corresponding graphical postfilter calcium concentration representation being distinct from all graphical representations of the remaining state parameters; and
rendering the postfilter calcium concentration representation in accordance with predefined postfilter calcium concentration animation rules.

In one embodiment, the method may further comprise:
receiving, from the data source, further time series of sampled measurement values for a systemic calcium concentration state parameter of the patient (arterial or venous blood gas analysis);
mapping the systemic calcium concentration state parameter to a predefined corresponding graphical systemic calcium concentration representation being distinct from all graphical representations of the remaining state parameters; and
rendering the systemic calcium concentration representation in accordance with predefined systemic calcium concentration animation rules.

In one embodiment, the method may further comprise:
receiving, from the data source, further time series of sampled anion gap state parameter values associated with unspecific anions;
mapping the anion gap state parameter to a predefined corresponding graphical anion gap representation being distinct from all graphical representations of the remaining state parameters; and
rendering the anion gap representation in relation to the arrangement for the hemofilter and patient's electrolyte state parameters in accordance with predefined anion gap animation rules.

In one embodiment, the method may further comprise:
receiving, from the data source, further time series of sampled low systemic calcium concentration and simultaneously increased anion gap state parameter values associated with unspecific anions, highlighting high risk of citrate accumulation,
mapping the citrate accumulation state parameter to a predefined corresponding graphical citrate accumulation representation being distinct from all graphical representations of the remaining state parameters; and
rendering the citrate accumulation representation in relation to the arrangement for the hemofilter and patient's electrolyte state parameters in accordance with predefined anion gap animation rules.

In one embodiment, the method may further comprise:
receiving, from the data source, further time series of sampled measurement values for a pH (H+ concentration, where equal to −log₁₀ c, where c is the hydrogen ion concentration in moles liter) and bicarbonate state parameters of the patient (arterial blood gas analysis);
mapping the concentration state parameters to a predefined corresponding graphical acid-base (metabolic acidosis, pH within normal limits or metabolic alkalosis) representation being distinct from all graphical representations of the remaining state parameters; and
rendering the acid-base representation in accordance with predefined acid-base state metabolic acidosis, pH within normal limits or metabolic alkalosis) animation rules.

In one embodiment, the method further comprises:
receiving, from the data source, further time series of sampled measurement values for Acid-Base Balance state parameters of the patient, comprising pH level and bicarbonate concentration,
mapping each Acid-Base Balance state parameter to a predefined corresponding graphical Acid-Base Balance representation being distinct from all graphical representations of the remaining state parameters; and
rendering, in the virtual 2D cross section of the blood-filled catheter inside scene representing the inside of the hemofilter catheter with blood flowing through the catheter and though the filter of the hemofilter, animated visualizations of the graphical Acid-Base Balance representations by using H+ and HCO3− ion objects and their ratio in accordance with predefined Acid-Base Balance animation rules.

Other representations may be chosen for the electrolyte objects by a person skilled in the art, such as for example, polygonal shaped objects instead of elliptic objects or droplet objects. Labelling of molecules and ions may also differ using different abbreviations, superscripts or subscripts for letters and numbers.

In a second aspect, a computer readable medium (computer program product) is provided comprising program instructions that, when loaded into a memory of a computing device and executed by at least one processor of the computing device, cause the at least one processor to execute the steps for comparing the following parameters: systemic calcium concentration from arterial or venous blood sample, postfilter calcium concentration from postfilter blood gas analysis, pH and bicarbonate ions from arterial blood gas analysis, against standard adjustment reference tables for calcium citrate hemofilter therapy. Outputs may include suggestions for therapy adjustments such as maintaining current blood flow parameters, increasing or decreasing blood flow parameters, maintaining current dialysate flow parameters, increasing or decreasing dialysate flow parameters, maintaining current citrate infusion rate parameters, increasing or decreasing citrate infusion parameters, maintaining current calcium infusion rate parameters, increasing or decreasing calcium infusion rate parameters, to support a medically trained person in monitoring and adjusting calcium citrate hemofilter therapy. The computer program has instructions which implement the rendering steps as described herein for the computer-implemented method. The computer program product includes at least instructions for:
receiving, from a data source, time series of sampled measurement values obtained from a plurality of blood gas analysis sensors for the following blood gas state parameters of the patient: systemic calcium concentration, sodium, potassium, chloride from arterial or venous blood sample, postfilter calcium concentration from postfilter blood gas analysis, pH and bicarbonate ions from arterial blood gas analysis.
receiving from a data source, time series of sampled measurement values obtained from a plurality of calcium citrate hemofilter analysis sensors for the following calcium citrate hemofilter state parameter settings: current calcium citrate hemofilter settings including blood flow, dialysate flow, citrate infusion rate, calcium infusion rate parameter settings,
relating each state parameter to predefined calcium citrate hemofilter therapy adjustment reference tables that can be individualized for specific patients, meaning comparing each parameter against reference tables, defining if the parameter is within normal range, too high or too low, mapping the conclusion to the recommended course of action as advised in the reference tables and generating outputs as predefined,
mapping each output to a predefined corresponding graphical representation with each graphical representation for a particular state parameter being distinct from all graphical representations of the remaining state parameters; and rendering, in a virtual blood-filled catheter inside scene representing the inside of the hemofilter catheter with blood flowing through it and though the filter of the calcium citrate hemofilter, animated visualizations of the graphical representations, in accordance with predefined animation rules, such that respective graphical objects move through the inside of the catheter and reflect current values of the respective state parameters.

In one embodiment, the method may further comprise:
receiving, from the data source, further time series of output measurement values for a blood flow parameter state of the calcium citrate hemofilter, which is a current setting of a blood flow in the calcium citrate hemofilter (Source 1) as well as blood gas analysis including pH, bicarbonate (Source 2), compared against standard reference tables;
mapping the blood flow parameter state of the calcium citrate hemofilter to a predefined corresponding graphical blood flow parameter state representation being distinct from all graphical representations of the remaining state parameters; and
rendering the blood flow parameter state representation in accordance with predefined blood flow parameter state animation rules.

In one embodiment, the method may further comprise:
receiving, from the data source, further time series of output measurement values for dialysate flow parameter state of the calcium citrate hemofilter, which is a current setting of a dialysate flow in the calcium citrate hemofilter (Source 1) as well as blood gas analysis including pH, bicarbonate (Source 2), compared against standard reference tables;
mapping the dialysate flow parameter state of the calcium citrate hemofilter to a predefined corresponding graphical dialysate flow parameter state representation being distinct from all graphical representations of the remaining state parameters; and
rendering the dialysate flow parameter state representation in accordance with predefined dialysate flow parameter state animation rules.

In one embodiment, the method may further comprise:
receiving, from the data source, further time series of output measurement values suggestions for citrate infusion rate parameter state of the calcium citrate hemofilter;
mapping the citrate infusion rate parameter state of the calcium citrate hemofilter to a predefined corresponding graphical citrate infusion rate parameter state representation being distinct from all graphical representations of the remaining state parameters; and
rendering the citrate infusion rate parameter state representation in accordance with the predefined citrate infusion rate parameter state animation rules.

In one embodiment, the method may further comprise:
receiving, from the data source, further time series of output measurement values suggestions for calcium infusion rate parameter state of the calcium citrate hemofilter;
mapping the calcium infusion rate parameter state of the calcium citrate hemofilter to a predefined corresponding graphical calcium infusion rate parameter state representation being distinct from all graphical representations of the remaining state parameters; and
rendering the calcium infusion rate parameter state representation in accordance with predefined calcium infusion rate parameter state animation rules.

In a third aspect, a computer system is provided for rendering representations of medical state parameters of a patient and calcium citrate hemofilter state parameters to support a medically trained person in calcium citrate hemofilter therapy management, comprising:
an interface adapted to receive, from a data source, time series of sampled measurement values obtained from a plurality of blood gas analysis sensors for the following blood gas state parameters of the patient: systemic calcium concentration from arterial or venous blood sample, postfilter calcium concentration from postfilter blood gas analysis, pH and bicarbonate ions from arterial blood gas analysis, and to receive data source, time series of sampled measurement values obtained from a plurality of calcium citrate hemofilter analysis sensors for the following calcium citrate hemofilter state parameter settings including blood flow, dialysate flow, citrate infusion rate, calcium infusion rate parameter settings,
an integrator module adapted to integrate received data from the blood gas analysis and current calcium citrate hemofilter settings with calcium citrate hemofilter therapy adjustment reference tables to generate suggestions for therapy adjustment data output,
a mapper module adapted to map each state parameter of blood gas analysis and therapy adjustment data output to a predefined corresponding graphical representation with each graphical representation for a particular state parameter or particular therapy adjustment suggestion being distinct from all graphical representations of the remaining state parameters and therapy adjustment suggestion; and,
a renderer module adapted to render, in the virtual blood-filled catheter inside scene representing the inside of the hemofilter catheter with blood flowing through it and though the filter of the calcium citrate hemofilter, animated visualizations of the graphical representations, in accordance with predefined animation rules, such that respective graphical objects move through the inside of the catheter and reflect current values of the respective state parameters and therapy adjustment recommendations.

Thereby, the predefined animation rules as disclosed herein can be used to display the animated visualizations of the graphical representations to a user (e.g., a medically trained person).

Further aspects of the description will be realized and attained by means of the elements and combinations particularly depicted in the appended claims. It is to be understood that both, the foregoing general description and the following detailed description are exemplary and explanatory only and are not restrictive of the description as described.

### BRIEF DESCRIPTION OF THE DRAWINGS

FIG. 1 shows a simplified diagram of a computer system for monitoring the clinical state of a patient during calcium citrate hemofilter therapy to support the selection of required adjustments to the calcium citrate hemofilter therapy according to an embodiment;

FIG. 2 is a simplified flowchart of a computer-implemented method for rendering representations of blood gas state parameters of a patient and calcium citrate therapy adjustment suggestion output according to an embodiment;

FIG. 3 illustrates a virtual 2D shaped scene representing the inside of a hemofilter blood catheter filled with blood flowing through it and through the filter including monitoring constituents and solutions relevant to therapy as rendered in accordance with an embodiment;

FIG. 4 illustrates a virtual 2D shaped scene representing the inside of a hemofilter blood catheter filled with blood flowing through it and through the filter including relevant to therapy monitoring constituents and solutions as rendered in accordance with an embodiment;

FIGS. 5A and 5B illustrate examples for rendering calcium citrate hemofilter therapy adjustment suggestion output results of dialysate flow objects for a dialysate flow rate state parameter in accordance with predefined animation rules according to an embodiment;

FIGS. 6A and 6B illustrate examples of rendering calcium citrate hemofilter therapy adjustment suggestion output results of citrate infusion objects for citrate infusion rate state parameter in accordance with predefined animation rules according to an embodiment;

FIGS. 7A and 7B illustrate examples of rendering calcium citrate hemofilter therapy adjustment suggestion output results of calcium infusion objects for calcium infusion rate state parameter in accordance with predefined animation rules according to an embodiment;

FIGS. 8A and 8B illustrate an example of rendering systemic calcium objects for a systemic calcium concentration state parameter in accordance with a respective predefined animation rule according to an embodiment;

FIGS. 9A to 9D illustrate an example of rendering postfilter calcium objects for a postfilter calcium concentration state parameter in accordance with a respective predefined animation rule according to an embodiment;

FIGS. 10 and 11 illustrate examples of rendering acid-base balance objects for various Acid-Base Balance state parameters in accordance with predefined animation rules according to an embodiment;

FIG. 12 illustrates an example of rendering citrate accumulation objects for various acid-base balance state parameters in accordance with predefined animation rules according to an embodiment;

FIG. 13 illustrates results from a Visual Hemofilter Study;

FIG. 14 illustrates study results regarding decision-making at the individual participant level; and

FIG. 15 is a diagram that shows an example of a generic computer device and a generic mobile computer device, which may be used with the techniques described here.

### DETAILED DESCRIPTION

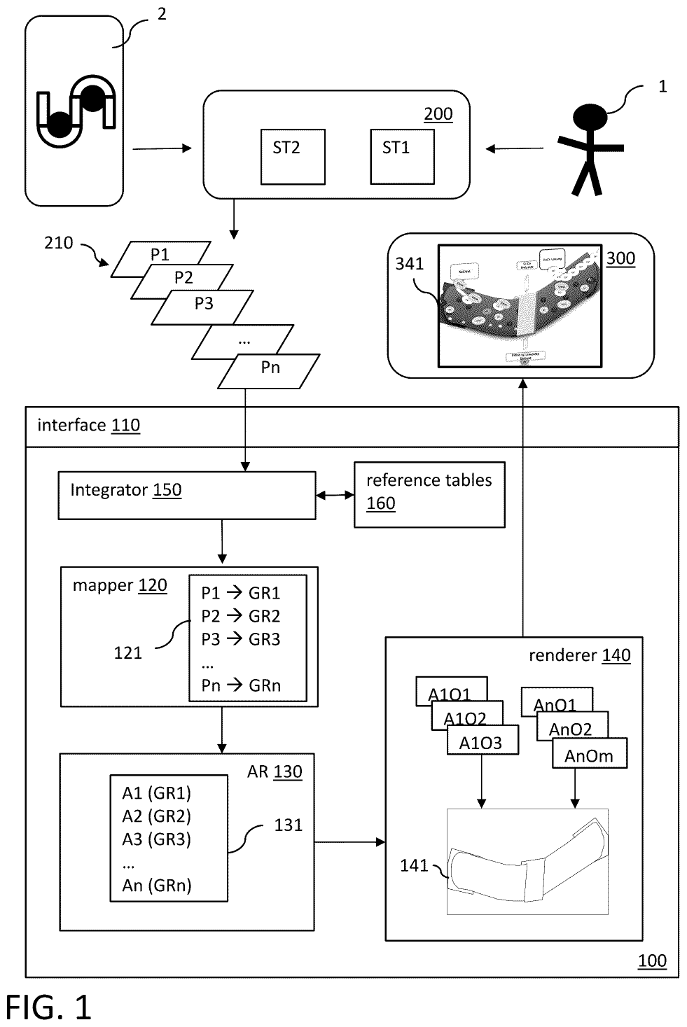

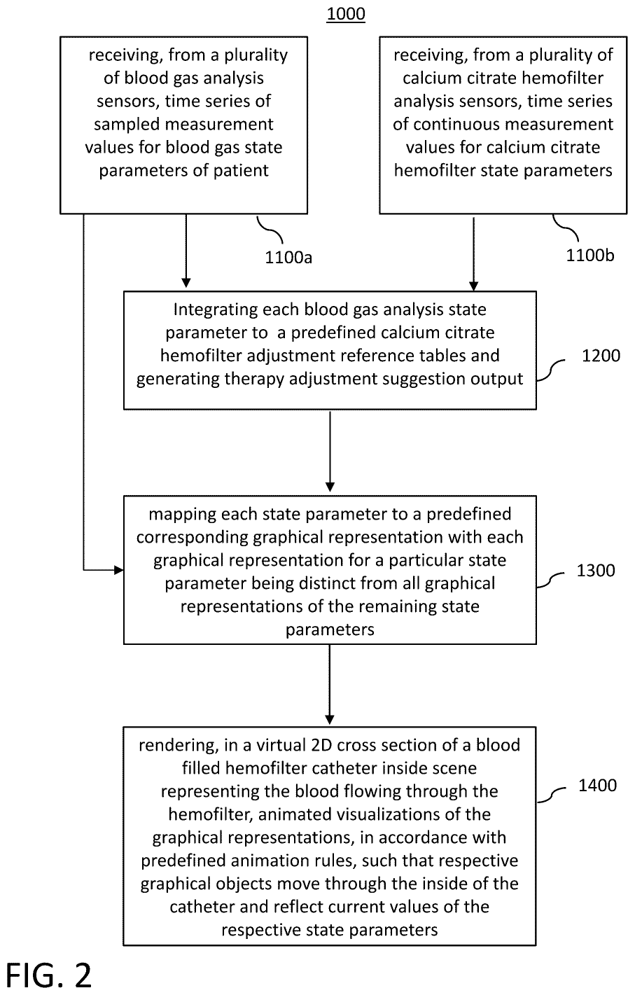

FIG. 1 shows a simplified diagram of an embodiment of a computer system 100 for monitoring the clinical state of a patient 1 during calcium citrate hemofilter therapy to support the selection of required adjustments to the calcium citrate hemofilter therapy. FIG. 2 is a simplified flowchart of a computer-implemented method 1000 for monitoring the clinical state of said patient 1 during calcium citrate hemofilter therapy to support the selection of required adjustments to the calcium citrate hemofilter therapy. The method 1000 can be performed by system 100. The following description describes the system 100 in the context of method 1000 and, therefore, refers to reference numbers of FIG. 1 and FIG. 2.

The system 100 is communicatively coupled with at least one data source 200 via interface 110. The data source 200 provides measured state parameter values P1 to Pn. Two types of state parameter values are provided. A first type ST1 of state parameters relates to time series of sampled measurement values obtained from a plurality of blood gas analysis sensors for the patient's 1 blood gas state parameters. Such blood gas state parameters comprise at least: systemic calcium concentration from arterial or venous blood sample, postfilter calcium concentration from postfilter blood gas analysis, pH and bicarbonate ions from arterial blood gas analysis. A second type ST2 of state parameters relates to time series of sampled measurement values obtained from a plurality of calcium citrate hemofilter analysis sensors (located in a calcium citrate hemofilter 2). The state parameters of the second type represent calcium citrate hemofilter state parameter settings comprising at least: blood flow, dialysate flow, citrate infusion rate, and calcium infusion rate parameter settings. It is to be noted that the at least one data source 200 may comprise multiple physical devices. For example, the blood gas state parameters may be directly obtained from respective blood gas sensors as data sources, and the hemofilter state parameters may be directly obtained from sensors of the hemofilter 2 or the hemofilter itself as data sources.

The blood gas state parameters of the patient may be obtained either directly by respective blood gas sensors, or may be computed based on respective sensor raw data. For example, the hemoglobin value of the patient is a blood gas state parameter (e.g., P1) which is directly obtained by a respective sensor. On the other hand, the anion gap of the patient is a blood gas parameter (e.g., P2) is computed based on other directly measured blood gas parameters. Typically, also computed blood gas parameters are provided by the data source 200. The one or more data sources may include the respective sensors and implement the necessary algorithm(s) to provide the measured and computed blood gas state parameters and hemofilter state parameters substantially in real time to system 100 while the patient 1 is being monitored by said sensors. However, the state parameters 210 may also be buffered by the one or more data sources 200 or even be stored in an intermediate storage device (not shown), and then be retrieved from the buffer or storage device by system 100 via an appropriate interface 110. The interface 110 may be any standard interface which is appropriate for the exchange of respective data.

The system 100 receives 1100a (via interface 110) from the one or more data sources, time series of sampled measurement values for blood gas state parameters of the patient 1. In a basic embodiment, the received time series include values for the blood gas parameters systemic calcium, postfilter calcium, pH, and bicarbonate. Further, system 100 receives 1100b, from a plurality of calcium citrate hemofilter analysis sensors of the hemofilter 2, time series of continuous measurement values for calcium citrate hemofilter state parameters. The blood gas time series and the hemofilter time series may be received via single data stream 210 as in the shown embodiment, or they may be received in parallel in separate data streams originating in respective data sources.

An integrator module 150 of system 100 integrates 1200 received blood gas analysis state parameter data and current calcium citrate hemofilter state parameters with calcium citrate hemofilter therapy adjustment reference tables 160 to generate suggestions for therapy adjustment data output. The calcium citrate hemofilter therapy adjustment reference tables 160. Those tables are predefined by the producer of the calcium citrate hemofilter. However, they may also be individually adjusted by a clinician depending on the patient's condition. A first table may include two columns: postfilter ionized calcium concentration (e.g., 0.36 mmol/L) and changes to citrate infusion that need to be performed depending on ionized calcium concentration (e.g., to increase the citrate dose by 0.1 mmol/L). A second table may include also two columns: systemic calcium concentration (e.g., 1.26 mmol/L) and changes to the calcium dose infused in the other column (e.g, to reduce the calcium dose by 0.2 mmol/L). Two tables/graphs may combine dialysate flow on the x-axis, blood flow on the y-axis and projections for different bicarbonate concentration.

A further example of therapy adjustment is a case of metabolic acidosis diagnosed in the blood gas of the patient. A clinical decision support tool can give individualized therapy adjustment recommendations for a patient to either increase the blood flow or decrease the dialysate flow, or both.

The received blood gas state parameter values and the generated therapy adjustment data outputs are then further processed by a mapper 120 module of system 100. The mapper 120 implements a mapping algorithm which maps 1200 each state parameter to a predefined corresponding graphical representation. For example, the blood flow parameter may be mapped to a graphic object which has the shape of a red blood cell. Of course, other graphical representations may be used. However, it is advantageous to use a graphical representation for a particular state parameter which is somehow associated with the respective state parameter. In any case, each graphical representation for a particular state parameter is distinct from all graphical representations of the remaining state parameters. Advantageously, distinct graphical representations are chosen such that the human visual system is able to distinguish the distinct graphical representations without a need for the human brain to perform complex mapping activities in the medically trained user's mental model which maps the graphical representations to the associated state parameters.

In the example embodiment of FIG. 1, the mapper 120 uses mapping rules 121 to map 1300 the state parameter P1 to a first graphical representation GR1, P2 to a second, distinct graphical representation GR2, P3 to a third, distinct graphical representation GR3 and so on until the last state parameter Pn is mapped to distinct graphical representation GRn. For the mapped graphical representations, the system has a set of predefined animation rules 131. The animation rules 131 are provided by an animation rule module AR 130. Thereby, for each graphical representation a subset of animation rules defines how the respective graphical object is to be rendered with animations that reflect the current value of the respective state parameter. For example, subset A1 includes one or more animation rules for the graphical representation GR1. Subset A2 includes one or more animation rules for the graphical representation GR2. Subset A3 includes one or more animation rules for the graphical representation GR3, and so on. Finally, subset An includes one or more animation rules for the graphical representation GRn. A subset of animation rules for a particular blood gas state parameter may, for example, include a first animation rule which reflects a normal state parameter value, a second animation rule which reflects a too high state parameter value, and a third animation rule which reflects a too low state parameter value. Some state parameters may be associated with only two states (e.g., normal, abnormal) in which case only two corresponding animation rules may be included in the respective subset. On the other hand, more than three states for a state parameter (e.g., critical high, too high, normal, too low, critical low) may also be supported in which case a corresponding animation rule for each state can be included in the respective subset.

In the example of the embodiment according to FIG. 1, the AR 130 module and the mapper 120 are integrated modules of system 100. However, these modules can also be provided by a remote device on a remote storage which is accessible by system 100 to retrieve the mapping 121 as well as the predefined animation rules 131 from the remote device.

A renderer module 140 of system 100 finally renders 1300 animated visualizations of the graphical representations in a virtual blood-filled catheter inside scene 141 representing the inside of a hemofilter catheter. The virtual scene 141 may be implemented as a virtual 2D cross section of the inside of the blood-filled catheter. In another implementation, a 3D visualization of the inside of the blood-filled catheter may be used as virtual scene 141 such that the blood flow through virtual scene can be visualized more realistically than when using a 2D virtual scene.

The animated visualizations 341 of the graphical representations are generated in accordance with the predefined animation rules, such that respective graphical objects A101, A102, A103, . . . , AnO1, AnO2, AnOm move through the inside of the blood-filled catheter with the hemofilter and reflect current values of the respective state parameters. Thereby, the graphical objects A101, A102, A103 represent three different animations as generated by the subset A1 of the predefined animation rules 131. The final rendering result 341 is then visualized on a display device 300 to the medically trained user (not shown). Of course, the result may also be displayed to the patient 1 in case the patient is in a condition which allows to look at the result. However, as shown in the description of the study below, in particular medically trained users benefit from the herein disclosed rendering result 341.

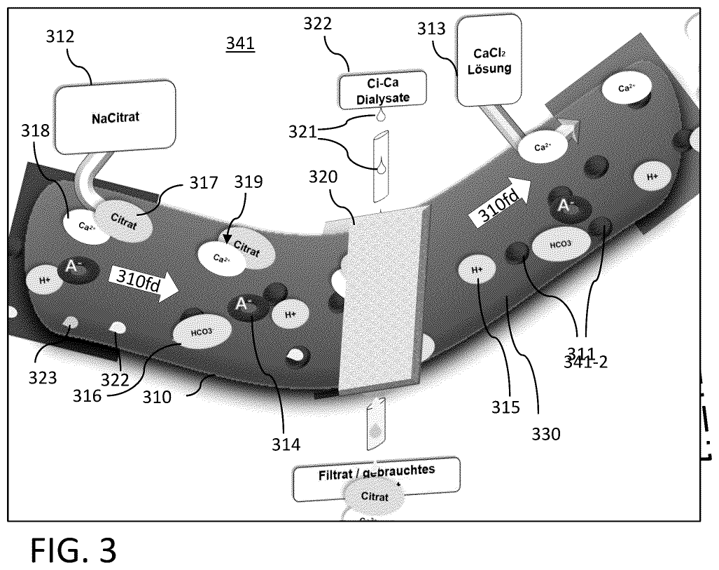

Turning briefly to FIG. 3, an example of a rendering result taken from an optional embodiment used in said study illustrates different animated graphical objects with each graphical object being a graphical representation of another state parameter. In this example, all graphical objects move at the same speed through the inside of the hemofilter catheter 310, 330. FIG. 3. illustrates a rendered virtual 2D scene 341 representing a cross-section of the inside of a hemofilter blood catheter 310, 330 (vessel) filled with blood flowing through it and through the filter 320 including monitoring constituents and solutions being relevant to therapy, where the visualized state parameters are within normal range. Blood flow is symbolized by red blood cell objects 311 with a shape resembling the shape of red blood cells moving in flow direction (from patient to filter, indicated by arrow 310fd) through the vessel 310 of catheter 330 and returning back to the patient. Dialysate flow is represented by droplet shaped objects 321 of a first type falling from a dialysate source object 322 into the filter 320. For example, the droplet objects of the first type may show a transparent coloring effect which is unique amongst all graphical representation objects. On the left is a citrate infusion object 312. Citrate molecules (represented by citrate objects 317) bind calcium ions (represented by calcium objects 318), forming calcium-citrate complexes (represented by calcium-citrate complexes object pairs 319). When all is within normal range, citrate molecules (represented by citrate objects 317), systemic calcium ions (represented by calcium objects 318) and calcium-citrate complexes (represented by calcium-citrate complexes object pairs 319) flow at similar rates (similar pace of animation), That is, the animated flow speeds of the objects 317, 318, 319 are substantially the same. Minor differences in the speed of individual objects may be acceptable as long as the animation of these objects creates the impression for the human visual system that the objects flow in sync through the vessel. On the right is a calcium infusion object 313. An A-ring object 314 symbolizes an anion gap. H+ objects 315 (hydrogen ion) and HCO₃⁻ objects 316 (bicarbonate ion) indicate acid-base hemostatic status.

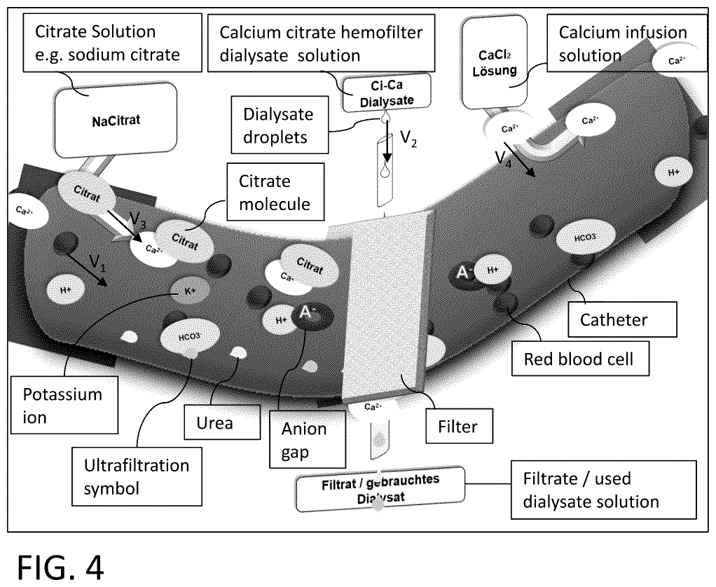

The predefined animation rules of the basic embodiment are now described in more detail by referring to FIGS. 4, 5, 6 and 7. The figures show screen shots of a version of the user interface that was used for the study described in detail further down below.

In the basic embodiment, the following predefined animation rules are used for animating the graphical representations of the blood flow rate, dialysate flow rate, citrate infusion rate and calcium infusion rate state parameters.

In FIG. 4, the different graphic representations moving through the inside of the blood filled hemofilter catheter are labelled with respective graphic object types according to their semantic meanings, i.e., with the medical parameters which are represented by the respective graphical object. Droplet shaped objects of a second type (cf. FIG. 3: urea symbol 322) indicating urea flowing in the vessel flow direction from the left-hand side of the blood-filled hemofilter catheter towards and through the filter represent the urea state of the patient. They are filtered out by the filter in the middle of the catheter visualized by changing the flow direction to vertical downwards (leaving the filter). In the following, droplet objects are simply referred to as droplets. Advantageously, the droplets of the second type differ from the droplets of the first type at least in color (e.g., using yellow color to represent urea). They may also differ in shape and size to make them clearly distinguishable for the human visual system. On average, two to four droplets of the second type entering the catheter on the left-hand side of the filter flow towards the filter indicating normal concentration of urea parameters. A high urea state is indicated if the number of urea objects is in the order of three times higher (approximately six to twelve urea droplet objects) flowing through the inside of the blood-filled catheter towards the filter at substantially the same speed as the rest of the blood constituent objects including red blood cells, potassium molecules (K⁺), hydrogen ion molecules (H⁺), and bicarbonate ion molecules (HCO₃⁻).

Droplets of a third type (cf. FIG. 3: ultrafiltration symbol 323) flowing in the vessel flow direction from the left-hand side of the blood-filled hemofilter catheter towards and through the filter, then changing direction of flow vertically downwards, indicate an ultrafiltration rate of the filter visualizing the removal of excess water from the patient's body through calcium citrate hemofiltration. Advantageously, the droplets of the third type differ from first/second type droplets at least in color (e.g., using blue color for representing water). In addition, they may also differ in shape and/or size to make them clearly distinguishable for the human visual system. On average two to four droplets of the third type entering on the left-hand side of the filter and flowing towards the filter indicate low to normal ultrafiltration rate. High ultrafiltration parameter setting is indicated if the number of droplets of the third type is in the order of three times higher (approximately six to twelve blue water molecule objects) flowing through the inside of the blood-filled catheter towards the filter at substantially the same speed as the rest of the blood constituent objects: red blood cells, potassium molecules (K⁺), hydrogen ion molecules (H⁺), bicarbonate ion molecules (HCO₃⁻).

Further, in FIG. 4, vectors V₁ to V₄ are used to explain animation rules for the pace of animation. Thereby, the vectors represent the relative pace of animation for the respective graphic objects. The blood flow rate V₁ is symbolized by red blood cells flowing through the blood catheter towards and through the filter. The dialysate flow rate V₂ is symbolized by droplets of the first type (e.g., transparent droplets) flowing downwards from the top of the user interface through the filter in the middle of the blood catheter. The citrate infusion rate V₃ is represented by citrate molecules flowing from the citrate solution in the vessel flow direction. The calcium infusion rate V₄ is symbolized by calcium ions (Ca²⁺) flowing from the calcium infusion solution on the right-hand side of the catheter in vessel flow direction. When the above hemofilter state parameter states are within normal limits, the respective rates of animation V₁ to V₄ are set to approximately 10 mm/sec.

In one animation rule example, a “too fast blood flow rate animation rule” suggests reduction of the blood flow rate V₁. In this example, the speed of the red blood cells representing the blood flow rate V₁ is in the order of three times faster compared to the speed of the red blood cells when the blood flow rate is within the recommended range. This also applies to the comparison with V₂, V₃ and V₄ when being within normal range.

In one animation rule example, a “too slow blood flow rate animation rule” suggests increase of V₁. In this example, V₁ is in the order of three times compared to the recommended range. This also applies to the comparison with V₂, V₃ and V₄ when being within normal range.

Blood flow objects may be shaped like red blood cells, the major constituent of blood, which facilitates the recognition of blood flow by the human visual system. Independent of object shape and labeling, the red blood cell objects are permanently visible (non-blinking) while moving through the inside of the hemofilter catheter.

In general, multiple single graphic objects of a given object type may move through the hemofilter catheter simultaneously. For example, a first object may move along the lower left side of the vessel and a second object of the same object type may move along the upper right side maintaining the same direction towards the right-hand side of the visualization screen. A new object of the same type may appear at the left-hand side of the virtual scene (e.g., a cross section of the hemofilter catheter), when a current object disappears from the view at the right-hand side of the hemofilter catheter visualization. Multiple objects of the same type may be rendered at different distances from each other, from the left and right-hand side of the cross section of the hemofilter catheter.

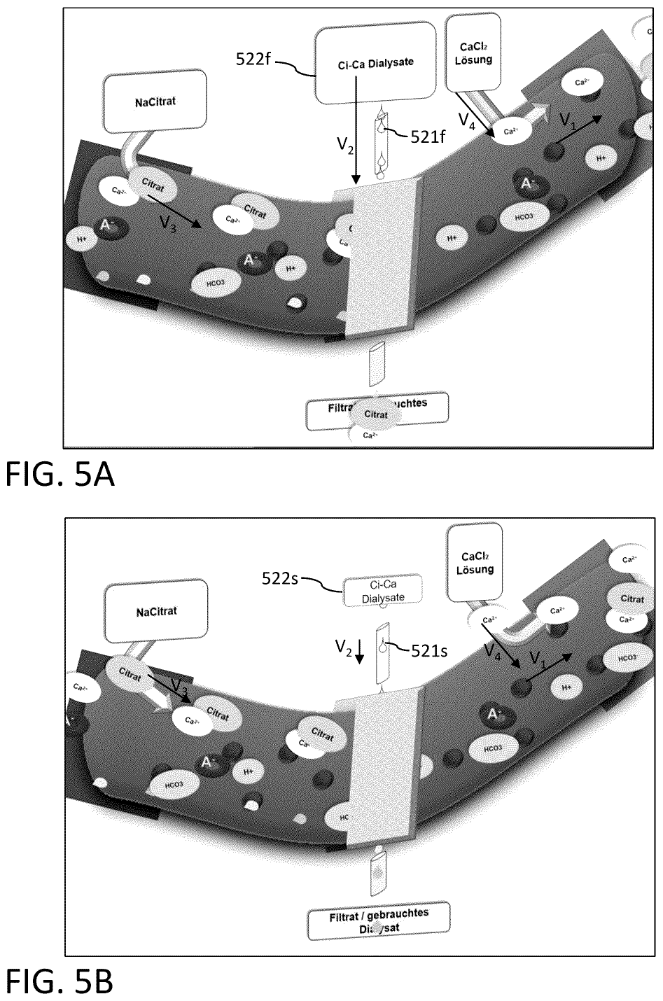

FIG. 5A illustrates an animation rule example of a “too fast dialysate flow rate V₂ animation rule” suggesting V₂ reduction. It is symbolized by (e.g., transparent) droplets of the first type 521f. flowing with a dialysate flow rate V₂ in the order of three times faster compared to the recommended range (and also compared to V₁, V₃ and V₄ when being within normal range). In one implementation of “too fast dialysate flow rate V₂ animation rule”, the calcium citrate hemofilter dialysate solution source 522f is also bigger than in the normal state (cf. object 322 in FIG. 3). Further, the edge of the dialysate solution source 522f may be enhanced by thickened contour line, when compared to the range being within normal limits as depicted in FIG. 3.

FIG. 5B illustrates an animation rule example of a “too slow dialysate flow rate V₂ animation rule” suggesting V₂ increase. In the example, dialysate flow rate pace of animation V₂ is in the order of three times slower compared to the recommended range (and compared to V₁, V₃ and V₄ when being within the normal range). In one implementation of this animation rule, the calcium citrate hemofilter dialysate solution source 521s may be smaller than in the normal state. Further, the edges of the dialysate solution source 522f may be displayed with a dashed-line contour.

Dialysate flow objects may be shaped like transparent droplets (of the second type), which facilitate the recognition of dialysate flow by the human visual system because Dialysate is also transparent in the real world. Independent of the object shape and labeling, the droplets of the second type are permanently visible (non-blinking) while falling into the filter.

In general, multiple single graphic objects of a given object type can move through the filter part of the hemofilter simultaneously in a vertical direction. A new object of the same type may appear at above the filter part of hemofilter, when a current object disappears from the view below the filter part of hemofilter. Multiple objects of the same type may be rendered at different distances from each other.

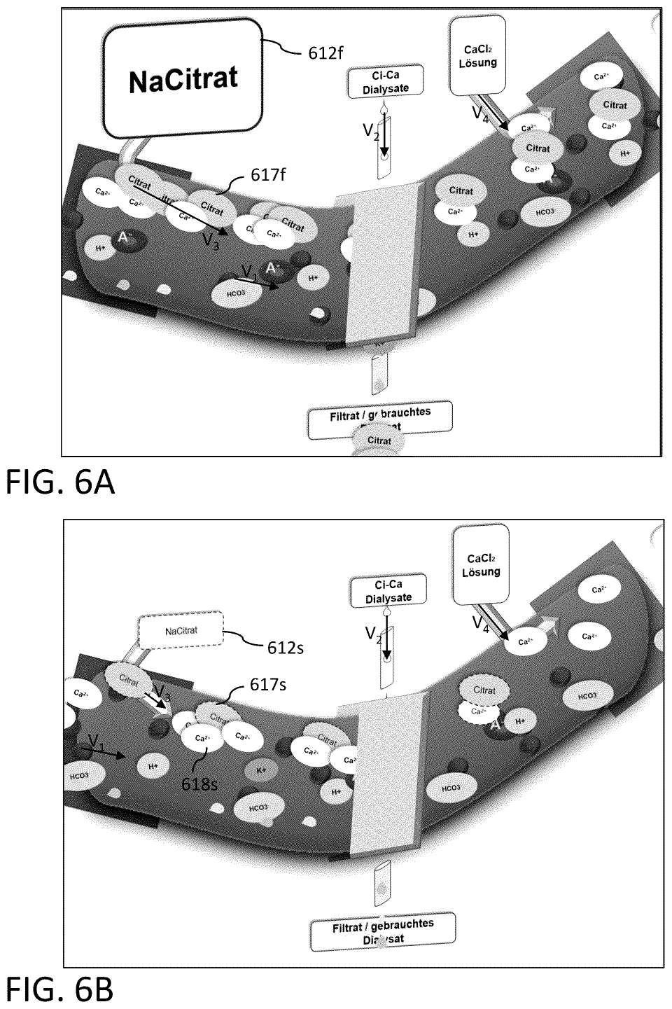

FIG. 6A illustrates an animation rule example of a “too fast citrate infusion rate V₃ animation rule” suggesting V₃ reduction. In the example, citrate infusion rate pace of animation V₃ is in the order of three times faster compared to the recommended range (and compared to V₁, V₂ and V₄, when being within normal range). In one implementation of this animation rule, the citrate infusion solution source object 612f is bigger compared to the normal range size. Further, the edges of object 612f may be enhanced by thickened contour line, when compared to objects representing parameters within normal limits.

In FIG. 6A, the number of citrate molecules is proportionally significantly higher than the number of calcium molecules whereas in a normal state, a ratio 1:1 is required to form calcium-citrate complexes. Therefore, the number of uncoupled citrate molecule objects 617f is higher compared to the normal state, thus indicating a too high citrate infusion rate V₃.

FIG. 6B illustrates an animation rule example of a “too slow citrate infusion rate animation rule” suggesting V₃ increase. In the example, the citrate infusion rate pace of animation V₃ is in the order of three times slower compared to the recommended range (and also compared to V₁, V₂ and V₄ when being within normal range. In one implementation, the calcium citrate hemofilter citrate solution source object 612s is also smaller compared to the normal state. Further, the contour of the infusion bag object 612s may be a dashed-line.

Citrate infusion objects may be shaped like citrate molecules that are labelled on every single individual molecule, which facilitate the recognition of citrate infusion objects by the human visual system. Citrate infusion objects bind calcium ions and form the citrate-calcium complexes as in human body, which are represented by a continuous overlapping of the respective graphic citrate and calcium objects to facilitate the recognition of citrate-calcium complexes by the human visual system. Independent of the object shape and labeling, the citrate-calcium complex objects are permanently visible (non-blinking) while moving through the inside of the hemofilter catheter.

As in the case of a too slow citrate infusion rate V₃ the number of citrate molecules is proportionally lower than the number of calcium molecules, the number of uncoupled systemic calcium molecules 618s is higher compared to the normal state, thus indicating too low citrate infusion rate V₃.

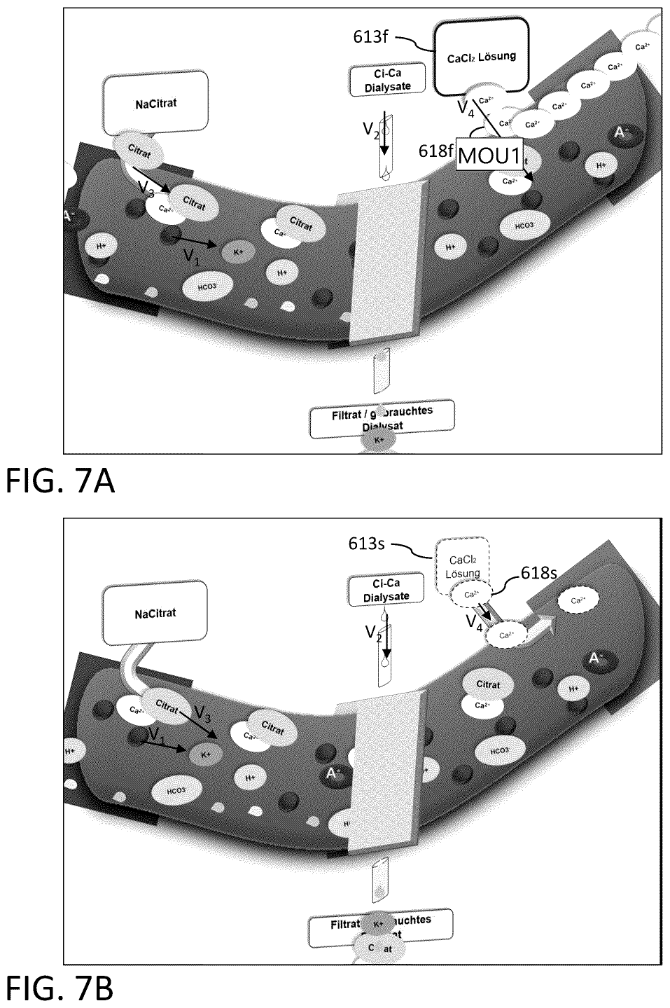

FIG. 7A illustrates an animation rule example of a “too fast calcium infusion rate animation rule” suggesting V₄ reduction. In the example, calcium (Ca²⁺ objects 618f) infusion rate pace of animation V₄ is in the order of three times faster compared to the recommended range (and also than V₁, V₂ and V₃, when being within normal range). In one implementation, the calcium infusion solution source object 613f is bigger compared to the normal range size. Further, the edges of object 613f may be enhanced by a thickened contour line.

FIG. 7B illustrates an animation rule example of a “too slow calcium infusion rate animation rule” suggesting V₄ increase. In the example, the calcium infusion rate pace of animation V₄ is in the order of three times slower compared to the recommended range (and also compared to V₁, V₂ and V₃ when being within normal range. In one implementation, the calcium infusion solution source object 613s is also smaller compared to the normal range size. Further, the contour of the infusion bag 618s of a solution may be a dashed-line.

Calcium infusion objects may be shaped like calcium molecules (Ca²⁺) that are labelled on every single individual molecule, which facilitate the recognition of calcium by the human visual system. Calcium ions are infused last just before returning circulation to the patient in daily clinical practice. Therefore, the respective calcium infusion objects 618s, 618f are also inserted into the visual hemofilter catheter representation at the end of the hemofilter catheter to facilitate the recognition of calcium infusion objects by the human visual system. Independent of the object shape and labeling, the calcium infusion objects are permanently visible (non-blinking) while moving through the inside of the hemofilter catheter.

In general, multiple single calcium infusion objects can simultaneously move through the hemofilter catheter after the filter part. A new calcium infusion object may appear to the right-hand side of the filter part of hemofilter, when a current object disappears from the scene. Multiple calcium infusion may be rendered at different distances from each other.

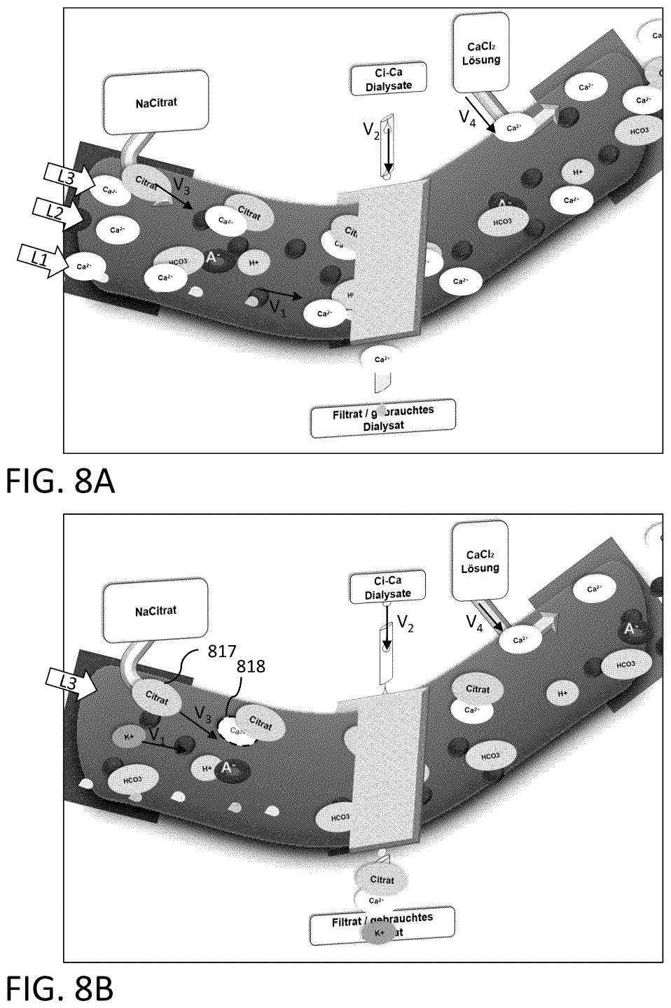

FIGS. 8A to 8B illustrate animation rules for the systemic calcium concentration state parameter.

FIG. 8A illustrates an animation rule example of an electrolyte animation rule subset for rendering multiple objects of the same object type flowing at multiple levels of the hemofilter catheter. In the example, a multilevel arrangement of calcium molecule objects (Ca²⁺) is shown on the left-hand side of the filter. The calcium molecule objects flow in three levels L1, L2, L3 through the vessel. This indicates a too high concentration of the respective electrolyte parameter systemic calcium concentration, Ca²⁺. For example, on average two calcium molecules on the left-hand side of the filter may flow towards the filter indicating normal concentration of the systemic calcium concentration parameters. If the number of calcium molecules is in the order of three times higher (e.g., approximately six calcium molecule objects) and such molecules flow through the inside of the blood-filled catheter towards the filter at substantially the same speed as the rest of the blood constituent objects (e.g., red blood cells, potassium molecules (K⁺), hydrogen ion molecules (H⁺), bicarbonate ion molecules (HCO₃⁻)), high systemic calcium concentration is indicated.

As the number of systemic calcium molecule objects is proportionally significantly higher than the number of citrate molecule objects, the number of uncoupled systemic calcium molecules is significantly higher, indicating too high systemic calcium concentration. In a normal state, a ratio 1:1 allows to form calcium-citrate complexes with all calcium molecule objects and citrate molecule objects.

FIG. 8B illustrates a further animation rule example of the electrolyte animation rule subset for rendering a single Ca²⁺ object 818 flowing at a single level L3 of the hemofilter catheter arrangement on the left-hand side of the filter. This example indicates a too low concentration of the respective electrolyte Ca²⁺ parameter: systemic calcium concentration. In the example implementation, the object 818 has a dashed line contour, indicating too low level of the respective electrolyte parameter. For example, on average two molecules on the left-hand side of the filter flowing towards the filter may indicate normal concentration of the systemic calcium concentration parameters. Low systemic calcium concentration is indicated if the number of calcium molecules is in the order of being halved (e.g., approximately only one calcium molecule object flowing through the inside of the blood-filled catheter towards the filter at substantially the same speed as the rest of the blood constituent objects: red blood cells, potassium molecules (K⁺), hydrogen ion molecules (H⁺), bicarbonate ion molecules (HCO₃⁻)). In this case, the number of uncoupled citrate molecules 817 is proportionally higher than the number of calcium molecules in the normal state, indicating too low systemic calcium concentration state because of a lack of calcium molecules required for forming calcium-citrate complexes.

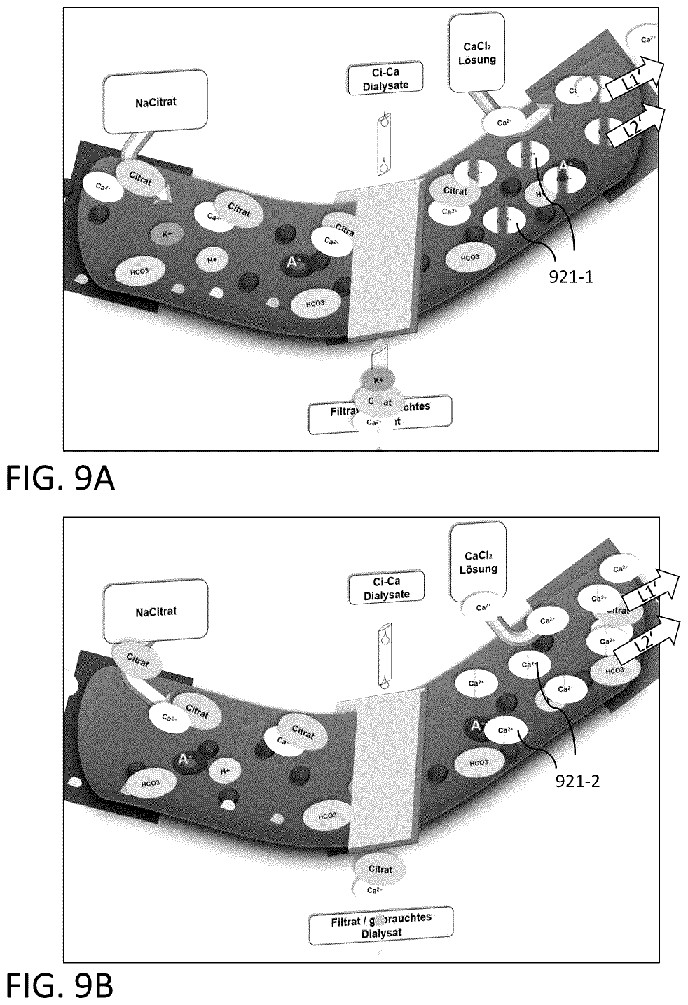

FIGS. 9A to 9D illustrate animation rules for the postfilter calcium concentration state parameter.

FIGS. 9A and 9B illustrates an animation rule example of an electrolyte animation rule subset for rendering multiple objects of the same object type appearing at multiple levels of the hemofilter catheter and not flowing with the rest of the blood constituents. The arrangement of calcium molecules (represented by exemplary calcium objects 921-1 and 921-2), labelled Ca²⁺, are blinking on at least two levels L1′ and L2′ on the right-hand side of the filter inside the hemofilter catheter. This indicates a too high concentration of the respective electrolyte state parameter postfilter calcium concentration, Ca²⁺. In general, if the postfilter calcium parameter is within normal range, there is no visualization of the postfilter calcium concentration. For example, if approximately six calcium molecule objects do not flow, but blink and appear on the right-hand side of the filter (postfilter) in case of left to right blood flow through the hemofilter catheter, the high postfilter calcium concentration is indicated. Animation of a respective calcium molecule object appearance can vary. In the example illustrated in FIGS. 9A and 9B, the object is animated such that its appearance starts from the edge of the molecule object and continues towards the inside of the object. Calcium objects 921-1 show an early stage of the animation at the beginning of its appearance and objects 921-2 show such molecule objects at a later stage of the animation, where the objects have almost completely appeared. For the postfilter calcium, one assumes the flow of the blood stream through the hemofilter catheter from the left towards the right-hand side of the visualization screen. Therefore, the postfilter electrolytes are shown on the right-hand side. If the flow is reverse, the postfilter objects would be on the left-hand side of the visualization screen.

FIGS. 9C and 9D illustrates an animation rule example of an electrolyte animation rule subset for rendering a single object for the low postfilter calcium concentration state parameter appearing at a single level of the hemofilter catheter and not flowing with the rest of the blood constituents. The animated singular object of a calcium molecule (represented by calcium objects 921-3 and 921-4), labelled Ca²⁺, is disappearing in the animation from the visualization in a blinking manner on the right-hand side of the filter inside the hemofilter catheter. In general, if the postfilter calcium state parameter is within normal range, there is no visualization of the postfilter calcium concentration. Animation of the calcium molecule appearance and animation of disappearance can vary. In the example shown in FIGS. 9C and 9D, the calcium object disappearance starts from its lower edge (post filter Ca²⁺ object 921-3) of and continues towards the upper edge of the molecule object (postfilter Ca²⁺ object 921-4). For the postfilter calcium, one assumes the flow of the blood stream through the hemofilter catheter from left towards right on the visualization screen. Therefore, the postfilter electrolytes are shown on the right-hand side. If the flow is reverse, the postfilter objects would be on the left-hand side of the visualization screen.

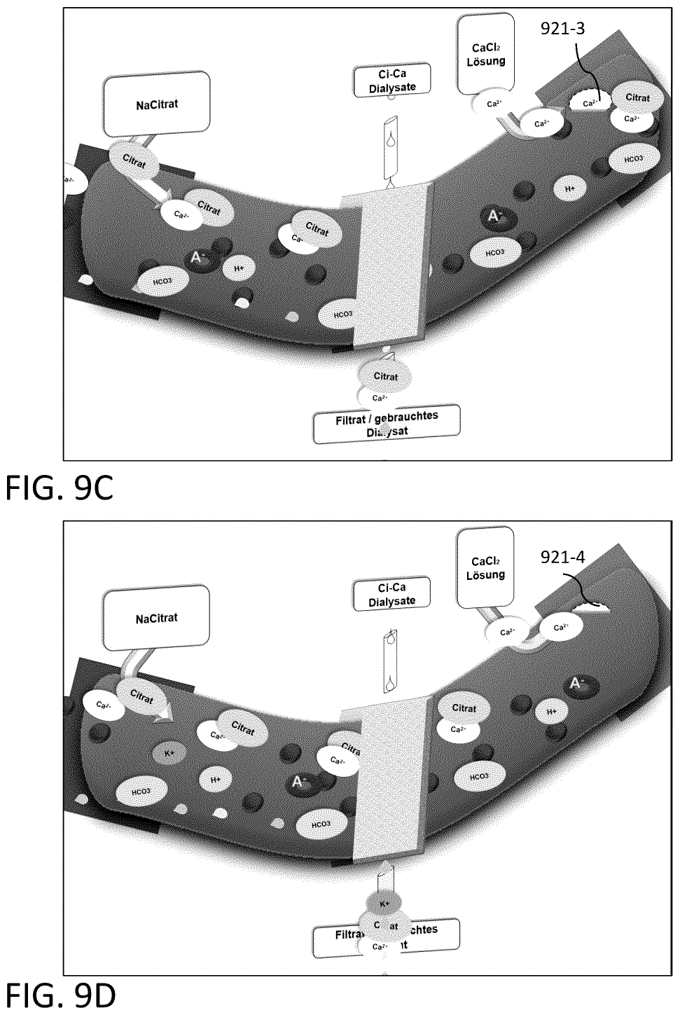

FIGS. 10 and 11 illustrate animation rules for the acid-base balance state parameter, acidosis and alkalosis respectively.

FIG. 10 illustrates an acid-base balance state parameter animation rule example of the acidosis. An electrolyte animation rule subset for rendering multiple objects flowing at multiple levels (levels L1″, L2″, L3″) of the hemofilter catheter arrangement of hydrogen ion objects 10-315 (labelled H⁺), on both sides of the filter, indicates a too high concentration of the respective electrolyte parameter and thus acidosis. In the example, on average two to four hydrogen molecules on both sides of the filter flow through the filter and indicate normal acid base status (cf. FIG. 3). Acidosis is indicated if the number of hydrogen molecules is two to four times higher, e.g., approximately four to eight hydrogen molecule objects flowing through the inside of the blood-filled catheter through the filter at substantially the same speed as the rest of the blood constituent objects (e.g., red blood cells, potassium molecules K⁺, calcium ion molecules). In the example, normal acid base status is indicated if, on average, two bicarbonate ions with a single thin continuous line contour (cf. FIG. 3, objects 316) flow through the filter at a single level in the blood stream. Acidosis is indicated if the same number of bicarbonate molecules flow at a single level of the catheter in the blood stream and have a dashed line contour as illustrated by objects 10-316r, wherein the number of bicarbonate ions is reduced proportionally to the number of hydrogen ions.

FIG. 11 illustrates an acid-base balance state parameter animation rule example of the alkalosis. For indicating a too high concentration of the respective electrolyte parameter (and thus alkalosis), an electrolyte animation rule renders multiple objects flowing at multiple levels (level L1′″, L2′″) of the hemofilter catheter arrangement of bicarbonate ions objects 10-316 (labelled HCO₃⁻) on both sides of the filter. For example, on average two to four bicarbonate molecules may flow on both sides and through the filter, indicating a normal acid base state (cf. FIG. 3). Alkalosis is indicated if the number of bicarbonate molecules is two to four times higher with approximately four to eight hydrogen molecule objects flowing through the inside of the blood-filled catheter through the filter at substantially the same speed as the rest of the blood constituent objects (e.g., red blood cells, potassium molecules (K⁺), calcium ion molecules). On average two hydrogen ions with single thin continuous line contour (cf. FIG. 3, objects 315) flow through the filter at a single level in the blood stream to indicate normal acid base state. If the same number of bicarbonate molecules (objects 10-315r) flow at a single level of the catheter in a blood stream and have a dashed line contour, the number of hydrogen ions is reduced proportionally to the number of bicarbonate ions and alkalosis is indicated.

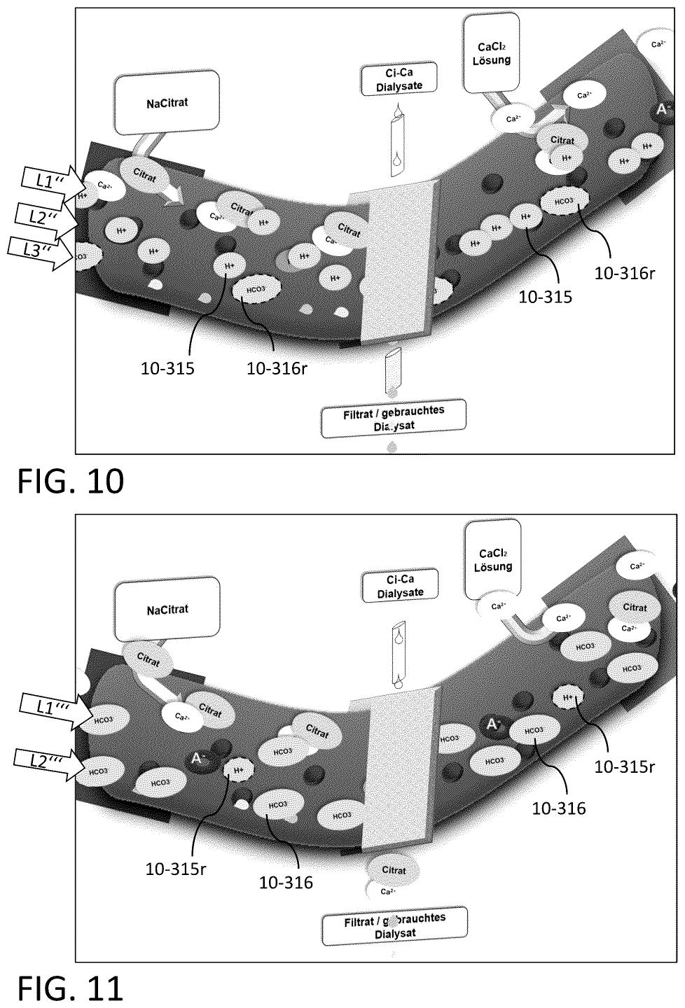

FIG. 12 illustrates a citrate accumulation using multiple animation rules.

Firstly, an animation of the anion gap relates to a doughnut shaped object 12-314 with a hole in the middle and labelled A−. An increased anion gap is indicated if doughnut shaped objects 12-314 increase in size and in number (e.g., by a factor of 2).

Secondly, an electrolyte animation rule renders a single object flowing at a single level of the hemofilter catheter arrangement (object 12-319 labelled Ca²⁺) on the left-hand side of the filter to indicate a too low concentration of the respective electrolyte parameter systemic calcium concentration (Ca²⁺). In the example, object 12-319 has a dashed line contour, indicating a too low level of the respective electrolyte parameter. In an example implementation, on average two molecule objects 12-319 on the left-hand side of the filter flowing towards the filter and coupling with corresponding citrate molecules maintaining a citrate/calcium ion ratio of 1:1, indicate normal concentration of the systemic calcium concentration parameters. If the number of calcium molecules flowing through the inside of the blood-filled catheter towards the filter is halved (e.g., approximately only one calcium molecule object), low systemic calcium concentration is indicated. This aspect together with an increased anion gap indicates a citrate accumulation state.

Thirdly, an additional sign for a citrate accumulation state is a blinking disappearing calcium molecule like object 12-318 (labelled Ca²⁺), with a dashed line contour that appears on the left-hand side of the visualization means (where systemic calcium ions usually enter the hemofilter catheter on the screen) and slowly disappears through respective animation.

Finally, multiple citrate-calcium bound complexes (as illustrated for example by object 12-340) at double the amount compared to a normal state (cf. FIG. 3.), flowing through the filter at multiple levels (e.g., level L1″″ and L2″″) are further additional signs that indicate citrate accumulation.

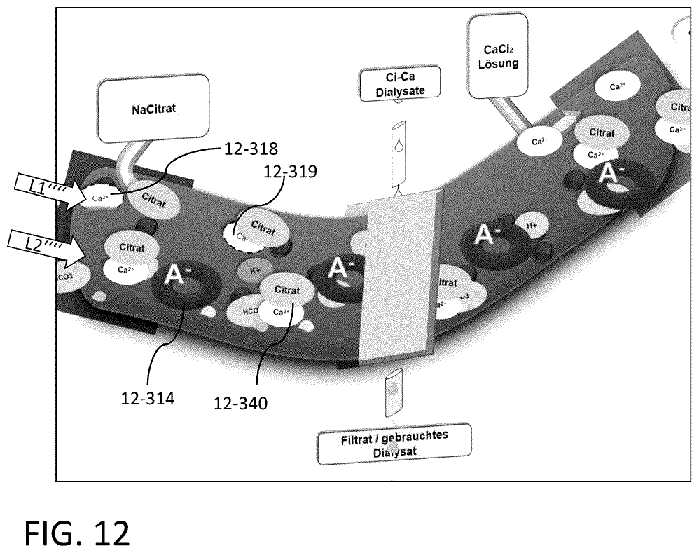

In FIG. 13, Total group differences between the conventional calcium citrate hemofilter reference table with standard blood gas analysis and the Visual Hemofilter. Box plots are medians with IQR, whiskers 5-95 percentiles, dots are individual outliers. Perceived diagnostic confidence: 0=very unconfident, 1=unconfident, 2=confident, 3=very confident. Perceived workload: NASA, National Aeronautics and Space Administration; TLX, task load index (scale 0-500); n=26. Statistical analysis performed with mixed logistic regression model for correct answers analysis and confidence rating (binary variable with the categories confident and unconfident. Mixed linear model was used for time to decision and perceived workload (NASA TLX score).

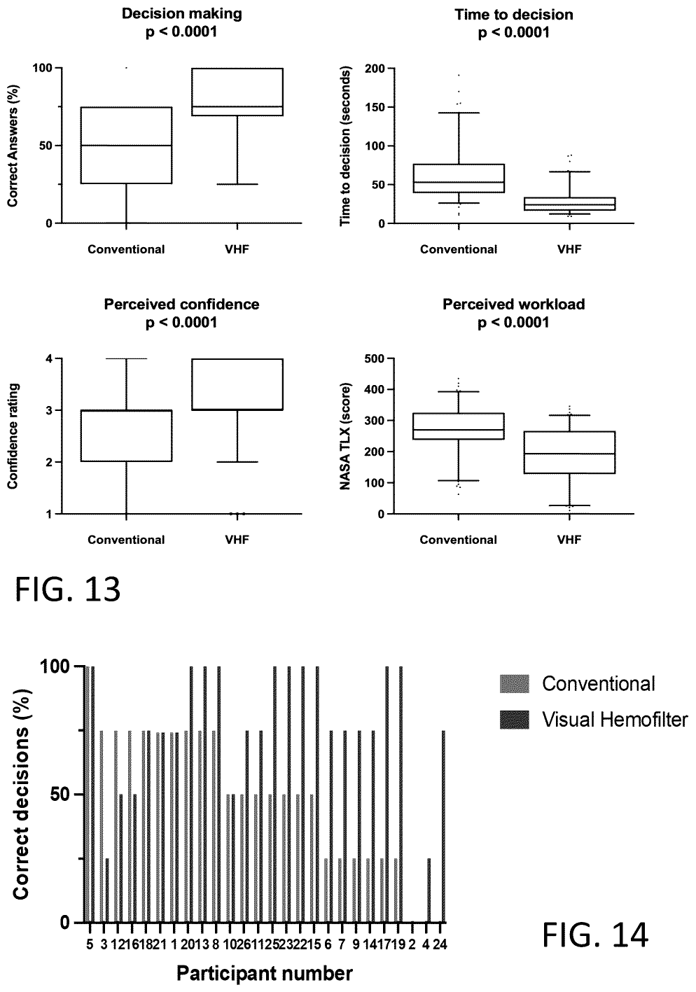

In FIG. 14, Decision-making at the individual participant level. Proportion of correct answers for each of the 26 participants ranked on the x-axis according to the proportion of correct decisions achieved with conventional calcium citrate hemofilter reference tables and standard blood gas analysis results printout.

### Study Results

The herein disclosed visualization of a Visual Hemofilter (VHF) is a novel information transfer technology and decision-support tool for the calcium citrate hemofilter, a recommended form of continuous renal replacement therapy for hemodynamically unstable patients. This technology animates the hemofilter's parameters and patient blood constituents as clearly distinguishable graphical objects flowing through the filter. Visual Hemofilter is designed to support critical care staff with their demanding workload, in particular, to optimize the information provided by blood gas results and calcium citrate hemofilter reference tables.

We conducted a prospective, randomized, computer-based simulation study in four intensive care units of the University Hospital Zurich. Twenty-six critical care professionals were presented with calcium citrate hemofilter scenarios, either as a Visual Hemofilter or with standard blood gas analysis and reference tables. They were then asked to make a therapy adjustment decision and rate their confidence and cognitive workload. We found that intensive care professionals were four times more likely to make correct decisions regarding calcium citrate hemofilter adjustments (odds ratio (OR) 3.96; 95% CI 2.03-7.73; p<0.0001 and made their decisions on average 33 seconds faster (−33.3; 95% CI −39.4-−27.2). They were also 5.4 times more likely to be confident in their decisions (OR 5.41; 95% CI 2.49-11.77; p<0.0001) and reported a significant 15% reduction in cognitive workload on a NASA TLX scale (−75.28; 95% CI −94.93-−55.63; p<0.0001).

By demonstrating that the Visual Hemofilter can lead to faster and more accurate decisions with higher confidence and lower cognitive workload, Visual Hemofilter assists critical care staff in their daily workload, increasing situational awareness and reducing the risk of potential errors.

### INTRODUCTION

Although the CaCi hemofilter has been shown to reduce the rate of complications compared to heparin, an increased rate of metabolic complications, particularly acid-base disturbances, has been associated with the CaCi hemofilter. Some of these complications could be prevented by prompt adjustment of therapy, highlighting the great potential for improvement supported by a visualization and decision support tool.

Time management is another problem that underscores the need for calcium citrate hemofilter visualization. The maintenance of the CaCi hemofilter, the monitoring of blood gas analysis and the necessary adjustments of the parameter settings are time-consuming. They require several steps and a high level of concentration. In fact, an Intensive Care Unit (ICU) nurse spends approximately 8 minutes per hour on CRRT. Easily accessible data with information presented in a single location in an understandable and user-centered manner assist users in making time-efficient decisions.

Patient blood gas analysis and postfilter blood gas analysis are the diagnostic standard to detect imbalances in patients' acid-base equilibrium and electrolyte status (systemic calcium and postfilter calcium) of the patient during calcium citrate hemofilter therapy. With the use of reference tables and two blood gas analyses (arterial blood gas and postfilter blood gas) one can monitor the patients state and adjust the therapy. The ability of modern blood gas analysis devices to measure a large variety of parameters from samples containing only a few milliliters of blood automatically, quickly, accurately and repeatedly represents a tremendous technological achievement. The well-established reference tables for monitoring acid-base patient status, systemic and postfilter calcium concentration and thus adjustments for calcium infusion, citrate infusion, blood flow and dialysate flow adjustments are fundamental for safe and efficient therapy management of a patient during hemofilter therapy.

However, to take full advantage of the specific strengths of the human perceptual system, prior art of blood gas analysis results in print outs. Calcium citrate hemofilter devices with their reference adjustment tables still have room for optimization with respect to how they present diagnostic information required for therapy monitoring and how they present adjustments required to be undertaken for further safe and effective hemofilter therapy. Prior arterial and postfilter blood gas result printouts include approximately 20-35 rows of tabular data, each including a parameter name, value, measurement unit, and expected normal range. The reference adjustment tables are predefined by the producer of the calcium citrate hemofilter. However, they can be individually adjusted by, for example, a clinician. An example of reference adjustment tables with first and second tables has already been described above. The reference adjustment tables typically vary depending on the manufacturer of the calcium citrate hemofilter, but in general, each table includes two columns and six rows. With regard to the graphs for blood flow and dialysate flow, users are required to have a substantial degree of familiarity of with such types of graphs and the respective interpretation of the data in the tables. Extracting information from such a printout requires caregivers to read and mentally translate a substantial quantity of textual and numerical data elements and integrate the derived meaning into their pre-existing mental models of calcium citrate hemofilter monitoring. In these mental constructs, they assign the various parameters to specific physiological functions or abnormalities, compare certain parameters against reference tables or graphs. These mental models vary widely among caregivers, which is reflected in the different orders in which caregivers read the parameters and in the different meanings they ascribe to them in different situations.

Moreover, this cognitively demanding process of interpretation happens in clinical environments where caregivers must deal with various factors that negatively affect their performance, such as information overload, distractions, and fatigue, which makes fostering situational awareness particularly challenging. Situational awareness is a three-step concept consisting of perceiving the relevant data elements in a situation, understanding their meaning, and projecting the situation's significance into the near future. High situational awareness allows to make decisions that are optimally adapted to a given situation and to perform appropriate actions. Research has identified situational awareness breakdowns as the primary cause of adverse events in anesthesia critical incident reporting system cases and malpractice claims and found strong evidence for a link between improving situational awareness and improving performance. For example, cognitive aids like the World Health Organization safety checklists improve situation awareness and outcomes, and situation awareness-oriented design improves diagnostic performance.

The animated visualization of the blood gas results and reference tables using the herein disclosed VHF approach optimizes the information presented in the blood gas results and reference tables for achieving situational awareness and supports clinical decision making by taking advantage of a visualization technique that allow the human visual system to easily extract decision relevant information from the user interface.

In this study, the results of a multimodal quantitative and survey study of VHF are reported. A test was performed on the primary hypothesis that participants make a higher number of correct diagnoses in simulated clinical scenarios with visual hemofilter than with conventional blood gas result printouts and reference tables. Further, the technology's effects on participants perceived diagnostic confidence and workload were examined. Furthermore, the learnability of VHF was examined and a survey to gain insights into users' perceptions and thus caregivers' acceptance of the concept was evaluated.

### Methods

The VHF version evaluated in this study is a software prototype simulating arterial blood and postfilter blood gas results for the users as well as outputs from the calcium citrate hemofilter reference tables and graphs, in a form of clinical decision suggestion for therapeutic adjustments.

VHF is an animation showing a virtual model of any given calcium citrate hemofilter situation by visualizing the arterial and postfilter parameters and their interactions as intuitive 2D graphical representation objects. Additionally, VHF integrates these results and compares them against reference adjustment tables and formulates clinical decision suggestions for calcium citrate hemofilter therapy maintenance. VHF was developed based on the principles of user-centered and situational awareness-oriented design. It follows the goal of situational awareness-oriented interface design: to convey the information needed by the caregivers as quickly as possible and with the lowest cognitive effort.

### Study Design and Participants

The study was a prospective, randomized, computer-based simulation study comparing two different methods of managing calcium citrate hemofilter therapy. The study was conducted across four different intensive care units at the University Hospital Zurich. Intensive care doctors in training (resident physicians), board-certified (staff physicians) and intensive care nurses were included. Participation in the study was voluntary, and participants received no financial compensation.

The study consisted of two parts. In the first part, it was investigated how well the participants assigned individual VHF visualizations to their corresponding problems with calcium citrate hemofilter after a short educational video. In the second part of the study, assessment of adjustments to the hemofilter's settings was performed with VHF and conventional methods. VHF outputs were compared with conventional blood result printouts and reference adjustment tables testing the hypothesis that using VHF enables participants to improve decision making with regards to the adjustments to the calcium citrate hemofilter, perceive higher diagnostic confidence and lower workloads.

### Study Procedure

The trial consisted of two parts. In part A, participants watched 15 randomized videos demonstrating a single anomaly. After each video, participants were asked to identify the anomaly from a selection of 15 potential answers (cf. Table 1). Each video lasted 45 seconds and participants had unlimited time to answer a question. This phase also aimed to introduce participants to this new visualization technology.

TABLE 1

| Nr | Single status/condition | Anomaly |
|---|---|---|
| 1 | Blood flow | too fast |
| 2 | Blood flow | too slow |
| 3 | Dialysate flow | too fast |
| 4 | Dialysate flow | too slow |
| 5 | Citrate concentration | high |
| 6 | Citrate concentration | low |
| 7 | Calcium infusion | too fast |
| 8 | Calcium infusion | too slow |
| 9 | Systemic calcium concentration | high |
| 10 | Systemic calcium concentration | low |
| 11 | Postfilter calcium concentration | high |
| 12 | Postfilter calcium concentration | low |
| 13 | Metabolic Acidosis | Hydrogen ions high, Bicarbonate ions low |
| 14 | Metabolic alkalosis | Bicarbonate ions low, Hydrogen ions low |
| 15 | Citrate accumulation | Increased anion gap & low systemic calcium |
| | | concentration |

Table 1: Visual Hemofilter scenarios single status/condition anomalies

Part B of the study involved assessment of adjustments to the hemofilter's settings. From a pool of eight possible scenarios, four were randomly selected for each participant. These scenarios showed both an anomaly and a required therapeutic adjustment. Each scenario was presented once using Visual Hemofilter and once using conventional blood gas analysis results with standard reference tables (cf. Table 2). The scenarios were presented in a random order for 45 seconds. After each case, participants were asked: “What is the appropriate course of action in this case?” with eight possible responses. The video was stopped or the blood gas analysis was removed 45 seconds after the start of the scenario or when the participant provided an answer. Participants had unlimited time to answer a question. Time to answer was recorded using the online data collection tool Harvest Your Data (Wellington, New Zealand). Having answered the main question for a scenario, participants rated their diagnostic confidence and perceived cognitive workload. The sequences in both parts were randomized before the start of the trial using Research Randomizer Version 4.0.

TABLE 2
Visual Hemofilter exemplary scenarios
Table 2: Visual Hemofilter exemplary scenarios.

| Nr | Scenario | Anomaly | Management |
|---|---|---|---|
| 1 | Systemic calcium | too high | Reduce the rate of |
| | concentration | | calcium infusion |
| 2 | Systemic calcium | too low | Increase the rate of |
| | concentration | | calcium infusion |
| 3 | Postfilter calcium | too high | Increase the rate of |
| | concentration | | citrate infusion |
| 4 | Postfilter calcium | too low | Decrease the rate of |
| | concentration | | citrate infusion |
| 5 | Metabolic | Hydrogen ions high, | Increase blood flow |
| | Acidosis | Bicarbonate ions low | |
| | | Blood flow too low | |
| 6 | Metabolic | Hydrogen ions high, | Reduce dialysate flow |
| | Acidosis | Bicarbonate ions low | |
| | | Dialysate flow too high | |
| 7 | Metabolic | Bicarbonate ions low, | Reduce blood flow |
| | alkalosis | Hydrogen ions low | |
| | | Blood flow too high | |
| 8 | Metabolic | Bicarbonate ions low, | Increase dialysate |
| | alkalosis | Hydrogen ions low | flow |
| | | Dialysate flow too low | |

### Study Outcome Measures

The primary outcome of the study was the decision-making endpoint, which was defined as the correct decision for each scenario in part B. In Visual Hemofilter scenarios, there was only one correct answer. However, in conventional scenarios, there were cases where two therapy adjustments were considered correct (e.g., metabolic acidosis scenario with two possible therapy adjustments: increase blood flow or reduce dialysate flow). In such cases, any of the correct answers provided was considered as correct. A decision was deemed correct if no incorrect answers were selected. All other therapy adjustments were considered incorrect, except in certain conventional scenarios where specific answers were not required, but the answer given was also not considered incorrect (e.g., metabolic acidosis: increase blood flow and increase citrate infusion rate, or vice versa). Time to decision was automatically measured in seconds. After each case, participants rated their diagnostic confidence using a four-point Likert scale, ranging from 1 (very unconfident) to 4 (very confident). Cognitive workload was assessed using the National Aeronautics and Space Administration Task Load Index (NASA-TLX), which consists of five questions scored on a scale of 0 to 100; the National Aeronautics and Space Administration Task Load Index (NASA-TLX; from 0-100) [Hart S G, Staveland L E. Development of NASA-TLX (Task Load Index): Results of Empirical and Theoretical Research. Advances in psychology 1988; 52:139-83.] We removed one question about “physical demand” that was irrelevant to our study, resulting in a total of five questions. In part A of the study, the interrater reliability for recognizing individual anomalies shown as Visual Hemofilter (parameter or condition) was calculated as the proportion of correctly identified anomalies per scenario. The learning effect was defined as the proportion of correctly identified anomalies based on the position order in which the scenarios were shown to the participant.

Before leaving, participants rated four general statements on a 5-point Likert scale (from strongly disagree to strongly agree) to capture their impressions about VHF. All responses were entered into a survey (Harvest your data, Wellington, New Zealand) on an iPad (Apple Inc., Cupertino, CA, USA).

### Statistical Analysis

For descriptive statistics, we calculated means, standard deviations, medians with interquartile ranges (IQR) for continuous data, and numbers with percentages for categorical data.

Univariate Poisson regression models were used to examine the associations between the number of correctly recognized parameters per participant and variables such as professional role, age, gender, work experience, and experience with the hemofilter.

To assess a learning effect, we used a mixed logistic regression model with a random intercept for each participant, considering the order of the videos shown as an influential variable. For the primary outcome analysis, we used McNemar's test, followed by a mixed logistic regression model with a random intercept per participant, adjusting for the corresponding scenario.

Time to decision and cognitive workload were analyzed using a mixed linear model. Confidence was analyzed by creating a binary variable with categories “confident” versus “not confident” and using a mixed logistic regression model. All mixed models included a random intercept for the participant and covariates such as modality and scenario name.

Statistical analyses were performed using R Version 4.0.5 (2021-03-31) (R Foundation for Statistical Computing, Vienna, Austria), and figures were generated using GraphPad PRISM 8.1.1. (GraphPad Software Inc., CA, USA). A p-value<0.05 was considered statistically significant.

### Sample Size Calculation

The sample size was calculated using data from a pilot study including six participants and the primary outcome, i.e., the proportion of correct therapy adjustment decisions with VHF and conventional measures. It was calculated that in order to construct a 95% confidence interval for an estimated proportion that extends no more than 10% in either direction, 26 participants are needed if the study power was set at 90%, i.e., 90% probability of correctly rejecting the null hypothesis that sample estimates which means detecting the difference between the groups.

### Results

Twenty-six intensive care doctors and nurses participated in the study and their baseline characteristics are summarized in Table 3.

TABLE 3
Table 3: Participant and study characteristics. Values are number (%), median
(range) or mean (SD). *when the conventional modality was shown in part B
of the study in the case of metabolic acidosis or alkalosis any correct answer
regarding blood flow or dialysate flow adjustment was marked as correct.

| Participant characteristics | Overall (N = 26) | |
|---|---|---|
| Gender; female | 17 | (65.4%) |
| Age (years) | | |
| Mean (SD) | 39.0 | (7.39) |
| Median [Min, Max] | 37 | [27.0, 57.0] |
| Role | | |
| Senior physician (%) | 5 | (19.2%) |
| Resident physician | 4 | (15.4%) |
| Nurse anesthetist | 16 | (61.5%) |
| Nurse anesthetist in training | 1 | (3.8%) |
| Job experience (years) | | |
| Mean (SD) | 13.8 | (7.78) |
| Median [Min, Max] | 14.0 | [1.00, 31.0] |
| Experience with Calcium Citrate Hemofilter (years) | | |
| Mean (SD) | 8.00 | (5.46) |
| Median [Min, Max] | 7.00 | [0, 23.0] |
| Study characteristics | | |
| Part A | | |
| 15 single anomaly videos shown as Visual Hemofilter | 390 | |
| Part B | | |
| Performed computer-based simulations | 208 | |
| shown as Visual Hemofilter | 104 | |
| Number of times each scenario was shown using each modality | | |

| | |
|---|---|
| High systemic calcium concentration | 11 |
| Low systemic calcium concentration | 11 |
| High postfilter calcium concentration | 16 |
| Low postfilter calcium concentration | 12 |
| Metabolic acidosis - Increase blood flow | 16 |
| Metabolic acidosis - Reduce dialysate flow | 17 |
| Metabolic alkalosis - Reduce blood flow | 12 |
| Metabolic alkalosis - Increase dialysate flow | 9 |

FIG. 13 illustrates total group differences between the conventional calcium citrate hemofilter reference table with standard blood gas analysis approach and the Visual Hemofilter approach. Box plots are medians with IQR, whiskers 5-95 percentiles, dots are individual outliers. Perceived diagnostic confidence: 0=very unconfident, 1=unconfident, 2=confident, 3=very confident. Perceived workload: NASA, National Aeronautics and Space Administration; TLX, task load index (scale 0-500); n=26. Statistical analysis performed with mixed logistic regression model for correct answers analysis and confidence rating (binary variable with the categories confident and unconfident. Mixed linear model was used for time to decision and perceived workload (NASA TLX score).

### Main Outcomes—Study Part 2

The use of Visual Hemofilter (VHF) significantly improved decision-making among intensive care professionals.

The use of Visual Hemofilter (VHF) significantly improved decision-making among intensive care professionals. The overall proportions (95% CI) of correct therapeutic decisions were 75% (63.9-86.1) for Visual Hemofilter and 49% (37.8-60.28) for the conventional modality. McNemar's test provided strong evidence for a difference between the two modalities, with an associated odds ratio of 3.45 (p<0.0002) in favour of Visual Hemofilter. Moreover, the mixed logistic regression model yielded an odds ratio of 3.96 (95% CI: 2.03-7.73; p<0.0001) for correct decisions with Visual Hemofilter compared to the conventional modality. This indicates that the odds of making the correct therapeutic decision were approximately four times higher when using Visual Hemofilter instead of standard measures. Individual participant analysis demonstrated that 18 out of 26 professionals performed better with Visual Hemofilter than with the conventional modality, with an additional five performing equally well with both modalities (FIG. 5). These findings underscore the significant advantage of Visual Hemofilter in enhancing decision-making accuracy among intensive care professionals.

FIG. 14 illustrates VHF decision-making results at the individual participant level. Proportion of correct answers for each of the 26 participants ranked on the x-axis according to the proportion of correct decisions achieved with conventional calcium citrate hemofilter reference tables and standard blood gas analysis results printout.

Intensive care professionals made decisions faster with Visual Hemofilter.

The mean time to reach a decision using conventional methods was 53 seconds (median 61.3; IQR 39.0-73.8; values range 11-191). However, when Visual Hemofilter was utilized, the mean time to decision reduced to 24 seconds (median 28.0; IQR 16.8-34.0; range 9-88). This significant improvement resulted in a 33-second reduction in the meantime to decision when using Visual Hemofilter compared to conventional methods. The mixed linear regression model demonstrated a Coefficient of −33.3 (95% CI −39.4-−27.2; p<0.0001), further affirming the time-saving advantage of Visual Hemofilter in the decision-making process.

Intensive care staff exhibited more decision confidence with Visual Hemofilter

In 27 out of 104 cases (26%), professionals described their confidence level as “very confident” when utilizing Visual Hemofilter, while only 14 out of 104 cases (13.5%) reported the same level of confidence with conventional methods. Additionally, a majority of participants rated their decision with Visual Hemofilter as “confident” in 60 out of 104 cases (57.7%), compared to 46 out of 104 cases (44.2%) with conventional methods. On the other hand, confidence level was described as “unsure” in 14 out of 104 cases (13.5%) with VHF, whereas with standard reference tables and blood gas analysis, this was the case in nearly a third of assessments (34 out of 104 cases; 32.7%). Similarly, only 3 out of 104 cases (2.9%) were reported as “unconfident,” and this was again higher with conventional methods (10 out of 104 cases; 9.6%). The odds of being confident about the decision made were 5.41 times higher when using Visual Hemofilter compared to conventional methods (OR 5.41; 95% CI 2.49-11.77; p<0.0001). This indicates a significant increase in decision confidence among intensive care staff when utilizing Visual Hemofilter.

Study participants reported a reduced cognitive workload when using Visual Hemofilter compared to conventional methods.

The mean NASA-TLX score, which is measured on a scale of 0 to 500, was 270 (median 267.1; IQR 239.2-323.0; values range 63-435) for the conventional modality. However, when Visual Hemofilter was employed, the mean score significantly decreased to 193 (median 191, IQR 130.5-266.2, variables range 11-346). The linear mixed model for cognitive workload provided robust evidence for a difference between the two modalities, revealing that using Visual Hemofilter resulted in a mean reduction of 75 points on the NASA Task Load Index (Coefficient −75.28; 95% CI −94.93-−55.63; p<0.0001).

The recognition of parameters in Visual Hemofilter improved throughout the study, indicating the presence of a moderate learning effect.

In part A of the study, a total of fifteen single-state or condition anomaly scenarios were tested. The mean number of correctly recognized parameters was 11.3 (SD 3.08), representing a recognition rate of 75.6% (SD 20.6%). The median number and percentage [range] of correctly recognized parameters by individuals were 12 [3-15], equivalent to 80% [20-100%]. The proportion (%) of correctly recognized parameters by scenario in part A is summarized in Table 4.

TABLE 4
Table 4: Proportion (%) of the correctly
recognized parameters by scenario; n = 26.

| | Proportion (%) of correctly recognized |
|---|---|
| | parameters/conditions |
| Part A scenario | in Part A |
| Blood flow too fast | 26/26 (100) |
| Calcium infusion too fast | 24/26 (92.3) |
| Citrate infusion too fast | 23/26 (88.5) |
| Metabolic alkalosis | 23/26 (88.5) |
| Blood flow too slow | 22/26 (84.6) |
| Dialysate flow too fast | 22/26 (84.6) |
| Metabolic acidosis | 22/26 (84.6) |
| Calcium infusion too slow | 21/26 (80.8) |
| Dialysate flow too slow | 21/26 (80.8) |
| Citrate accumulation | 18/26 (69.2) |
| Systemic [Calcium] too low | 17/26 (65.4) |
| Postfilter [Calcium] too high | 16/26 (61.5) |
| Citrate infusion too slow | 15/26 (57.7) |
| Systemic [Calcium] too high | 15/26 (57.7) |
| Postfilter [Calcium] too high | 10/26 (38.5) |

The study investigated various covariates such as age, gender, professional role, job experience, and experience with Calcium Citrate Hemofilter. However, no significant association was found between parameter recognition and these covariates (univariate Poisson regression models; p-value>0.05).

Furthermore, a mixed logistic regression model provided moderate evidence of a learning effect. With each “repetition” of scenarios, the odds of solving the scenario correctly increased by a factor of 1.06 (CI 1.00-1.13; p-value 0.047) (FIG. 6). This suggests that as participants engaged with Visual Hemofilter scenarios more frequently, their ability to correctly identify parameters improved, indicating a learning effect over time.

### DISCUSSION

This multimodal study investigated VHF, a technology visualizing calcium citrate hemofilter current settings, relevant blood gas results for monitoring of the patient during hemofilter therapy and therapy adjustment suggestions. It was developed to improve caregivers' situation awareness and improve monitoring and prompt and adequate adjustments to the calcium citrate hemofilter settings. The study found that intensive care professionals were four times more likely to make correct decisions regarding calcium citrate hemofilter adjustments and made their decisions faster. They were also more likely to be confident in their decisions and reported a significant reduction in cognitive workload. Furthermore, the participants could easily learn to recognize the visualizations from a short educational video and rated their experience positively.

The substantial effect size observed to date with these technologies demonstrates their significant potential to improve decision-making.

In this study, we also found that VHF improved perceived diagnostic confidence, was easy to learn and was most helpful to participants who had fewer correct decisions for therapy adjustment with the conventional blood gas result printout and standard reference tables. These results suggest that VHF helps to shorten new caregivers' education times and enables all members of a care team to participate in clinical decision-making. This aspect is particularly important as projections from the World Health Organization are for ever-increasing patient numbers, case complexity, and a global shortage of healthcare professionals.

The underlying mechanism for the improvements found with VHF appears to be in its situational awareness-oriented design. For example, in the VHF study, when calcium infusion was flowing at a higher rate, it was indicated that the calcium infusion rate is too high and should be reduced. This representation reduces caregivers' cognitive effort, as the translation and classification of text abbreviations and associated numbers and their integration into caregivers' own mental model are no longer necessary. Streamlining this mental model creation process also results in the improved confidence and reduced perceived workload observed with VHF.

VHF is designed to leave the final diagnosis and therapy decisions to the human decision-makers. The caregivers are provided with visualizations to efficiently present the information but not to make a definitive diagnosis for them, as would be the case with a text output of the diagnosis. So VHF is used as a support tool for the caregivers supporting them in making fast and correct therapy adjustment decisions.

### CONCLUSIONS

In conclusion, we found that intensive care physicians and nurses were four times more likely to make correct calcium citrate hemofilter adjustment decisions when Visual Hemofilter was used, and they made their decisions on average 33 seconds faster. They were also 5.4 times more likely to be confident in their decision and reported a 15% reduction in cognitive workload, as measured by the NASA-TLX score.

By demonstrating in this randomized, computer-based simulation study that Visual Hemofilter leads to faster and more correct decisions with higher confidence and lower cognitive workload, we are one step closer to supporting critical care staff in their workload, increasing situational awareness and reducing the risk of potential errors. Further studies are needed to evaluate critical care staff performance with Visual Hemofilter in a high-fidelity simulation suite and, next, in clinical practice.

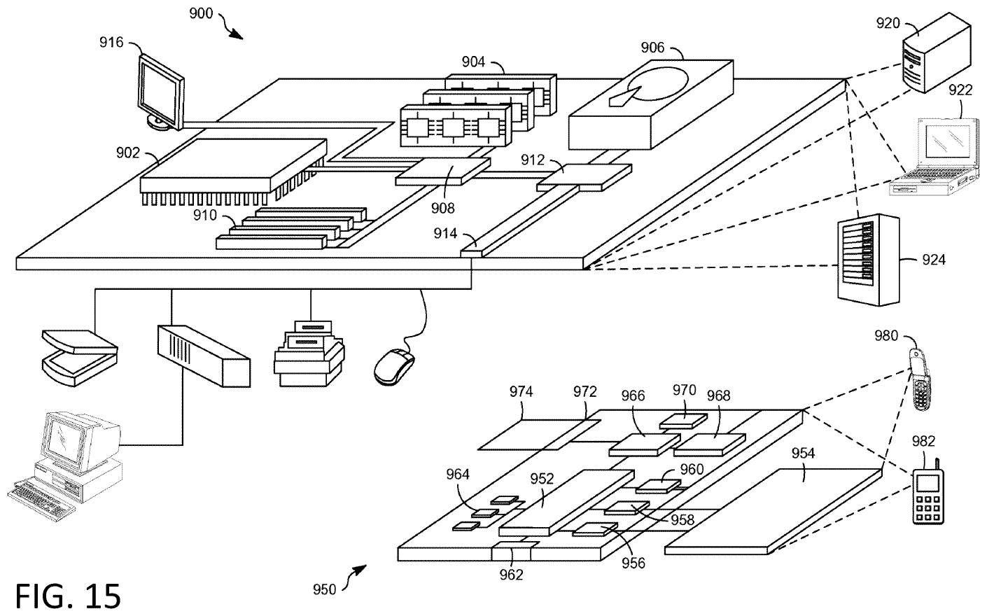

FIG. 15 is a diagram that shows an example of a generic computer device 900 and a generic mobile computer device 950, which may be used with the techniques described here. Computing device 900 is intended to represent various forms of digital computers, such as laptops, desktops, workstations, personal digital assistants, servers, blade servers, mainframes, and other appropriate computers. Generic computer device 900 may correspond to the computer system 100 of FIG. 1 for monitoring the clinical state of a patient during calcium citrate hemofilter therapy to support the selection of required adjustments to the calcium citrate hemofilter therapy. Computing device 950 is intended to represent various forms of mobile devices, such as personal digital assistants, cellular telephones, smart phones, and other similar computing devices. For example, computing device 950 may be used as a frontend by a user (e.g., medically trained staff) to interact with the computing device 900. For example, the user may receive real-time monitoring data about the health state of a particular patient which requires the adjustment of one or more hemofilter state parameters. The components shown here, their connections and relationships, and their functions, are meant to be exemplary only, and are not meant to limit implementations described and/or claimed in this document.

Computing device 900 includes a processor 902, memory 904, a storage device 906, a high-speed interface 908 connecting to memory 904 and high-speed expansion ports 910, and a low speed interface 912 connecting to low speed bus 914 and storage device 906. Each of the components 902, 904, 906, 908, 910, and 912, are interconnected using various busses, and may be mounted on a common motherboard or in other manners as appropriate. The processor 902 can process instructions for execution within the computing device 900, including instructions stored in the memory 904 or on the storage device 906 to display graphical information for a GUI on an external input/output device, such as display 916 coupled to high speed interface 908. In other implementations, multiple processors and/or multiple buses may be used, as appropriate, along with multiple memories and types of memory. Also, multiple computing devices 900 may be connected, with each device providing portions of the necessary operations (e.g., as a server bank, a group of blade servers, or a multi-processor system).

The memory 904 stores information within the computing device 900. In one implementation, the memory 904 is a volatile memory unit or units. In another implementation, the memory 904 is a non-volatile memory unit or units. The memory 904 may also be another form of computer-readable medium, such as a magnetic or optical disk.

The storage device 906 is capable of providing mass storage for the computing device 900. In one implementation, the storage device 906 may be or contain a computer-readable medium, such as a floppy disk device, a hard disk device, an optical disk device, or a tape device, a flash memory or other similar solid-state memory device, or an array of devices, including devices in a storage area network or other configurations. A computer program product can be tangibly embodied in an information carrier. The computer program product may also contain instructions that, when executed, perform one or more methods, such as those described above. The information carrier is a computer- or machine-readable medium, such as the memory 904, the storage device 906, or memory on processor 902.

The high-speed controller 908 manages bandwidth-intensive operations for the computing device 900, while the low speed controller 912 manages lower bandwidth-intensive operations. Such allocation of functions is exemplary only. In one implementation, the high-speed controller 908 is coupled to memory 904, display 916 (e.g., through a graphics processor or accelerator), and to high-speed expansion ports 910, which may accept various expansion cards (not shown). In the implementation, low-speed controller 912 is coupled to storage device 906 and low-speed expansion port 914. The low-speed expansion port, which may include various communication ports (e.g., USB, Bluetooth, Ethernet, wireless Ethernet) may be coupled to one or more input/output devices, such as a keyboard, a pointing device, a scanner, or a networking device such as a switch or router, e.g., through a network adapter.

The computing device 900 may be implemented in a number of different forms, as shown in the figure. For example, it may be implemented as a standard server 920, or multiple times in a group of such servers. It may also be implemented as part of a rack server system 924. In addition, it may be implemented in a personal computer such as a laptop computer 922. Alternatively, components from computing device 900 may be combined with other components in a mobile device (not shown), such as device 950. Each of such devices may contain one or more of computing device 900, 950, and an entire system may be made up of multiple computing devices 900, 950 communicating with each other.

Computing device 950 includes a processor 952, memory 964, an input/output device such as a display 954, a communication interface 966, and a transceiver 968, among other components. The device 950 may also be provided with a storage device, such as a Microdrive or other device, to provide additional storage. Each of the components 950, 952, 964, 954, 966, and 968, are interconnected using various buses, and several of the components may be mounted on a common motherboard or in other manners as appropriate.

The processor 952 can execute instructions within the computing device 950, including instructions stored in the memory 964. The processor may be implemented as a chipset of chips that include separate and multiple analog and digital processors. The processor may provide, for example, for coordination of the other components of the device 950, such as control of user interfaces, applications run by device 950, and wireless communication by device 950.

Processor 952 may communicate with a user through control interface 958 and display interface 956 coupled to a display 954. The display 954 may be, for example, a TFT LCD (Thin-Film-Transistor Liquid Crystal Display) or an OLED (Organic Light Emitting Diode) display, or other appropriate display technology. The display interface 956 may comprise appropriate circuitry for driving the display 954 to present graphical and other information to a user. The control interface 958 may receive commands from a user and convert them for submission to the processor 952. In addition, an external interface 962 may be provide in communication with processor 952, so as to enable near area communication of device 950 with other devices. External interface 962 may provide, for example, for wired communication in some implementations, or for wireless communication in other implementations, and multiple interfaces may also be used.

The memory 964 stores information within the computing device 950. The memory 964 can be implemented as one or more of a computer-readable medium or media, a volatile memory unit or units, or a non-volatile memory unit or units. Expansion memory 984 may also be provided and connected to device 950 through expansion interface 982, which may include, for example, a SIMM (Single In Line Memory Module) card interface. Such expansion memory 984 may provide extra storage space for device 950, or may also store applications or other information for device 950. Specifically, expansion memory 984 may include instructions to carry out or supplement the processes described above, and may include secure information also. Thus, for example, expansion memory 984 may act as a security module for device 950, and may be programmed with instructions that permit secure use of device 950. In addition, secure applications may be provided via the SIMM cards, along with additional information, such as placing the identifying information on the SIMM card in a non-hackable manner.

The memory may include, for example, flash memory and/or NVRAM memory, as discussed below. In one implementation, a computer program product is tangibly embodied in an information carrier. The computer program product contains instructions that, when executed, perform one or more methods, such as those described above. The information carrier is a computer- or machine-readable medium, such as the memory 964, expansion memory 984, or memory on processor 952 that may be received, for example, over transceiver 968 or external interface 962.

Device 950 may communicate wirelessly through communication interface 966, which may include digital signal processing circuitry where necessary. Communication interface 966 may provide for communications under various modes or protocols, such as GSM voice calls, SMS, EMS, or MMS messaging, CDMA, TDMA, PDC, WCDMA, CDMA2000, or GPRS, among others. Such communication may occur, for example, through radio-frequency transceiver 968. In addition, short-range communication may occur, such as using a Bluetooth, WiFi, or other such transceiver (not shown). In addition, GPS (Global Positioning System) receiver module 980 may provide additional navigation- and location-related wireless data to device 950, which may be used as appropriate by applications running on device 950.

Device 950 may also communicate audibly using audio codec 960, which may receive spoken information from a user and convert it to usable digital information. Audio codec 960 may likewise generate audible sound for a user, such as through a speaker, e.g., in a handset of device 950. Such sound may include sound from voice telephone calls, may include recorded sound (e.g., voice messages, music files, etc.) and may also include sound generated by applications operating on device 950.

The computing device 950 may be implemented in a number of different forms, as shown in the figure. For example, it may be implemented as a cellular telephone 980. It may also be implemented as part of a smart phone 982, personal digital assistant, or another similar mobile device.

Various implementations of the systems and techniques described here can be realized in digital electronic circuitry, integrated circuitry, specially designed ASICs (application specific integrated circuits), computer hardware, firmware, software, and/or combinations thereof. These various implementations can include implementation in one or more computer programs that are executable and/or interpretable on a programmable system including at least one programmable processor, which may be special or general purpose, coupled to receive data and instructions from, and to transmit data and instructions to, a storage system, at least one input device, and at least one output device.

These computer programs (also known as programs, software, software applications or code) include machine instructions for a programmable processor, and can be implemented in a high-level procedural and/or object-oriented programming language, and/or in assembly/machine language. As used herein, the terms “machine-readable medium” and “computer-readable medium” refer to any computer program product, apparatus and/or device (e.g., magnetic discs, optical disks, memory, Programmable Logic Devices (PLDs)) used to provide machine instructions and/or data to a programmable processor, including a machine-readable medium that receives machine instructions as a machine-readable signal. The term “machine-readable signal” refers to any signal used to provide machine instructions and/or data to a programmable processor.

To provide for interaction with a user, the systems and techniques described here can be implemented on a computer having a display device (e.g., a CRT (cathode ray tube) or LCD (liquid crystal display) monitor) for displaying information to the user and a keyboard and a pointing device (e.g., a mouse or a trackball) by which the user can provide input to the computer. Other kinds of devices can be used to provide for interaction with a user as well; for example, feedback provided to the user can be any form of sensory feedback (e.g., visual feedback, auditory feedback, or tactile feedback); and input from the user can be received in any form, including acoustic, speech, or tactile input.

The systems and techniques described here can be implemented in a computing device that includes a back end component (e.g., as a data server), or that includes a middleware component (e.g., an application server), or that includes a front end component (e.g., a client computer having a graphical user interface or a Web browser through which a user can interact with an implementation of the systems and techniques described here), or any combination of such back end, middleware, or front end components. The components of the system can be interconnected by any form or medium of digital data communication (e.g., a communication network). Examples of communication networks include a local area network (“LAN”), a wide area network (“WAN”), and the Internet.

The computing device can include clients and servers. A client and server are generally remote from each other and typically interact through a communication network. The relationship of client and server arises by virtue of computer programs running on the respective computers and having a client-server relationship to each other.

A number of embodiments have been described. Nevertheless, it will be understood that various modifications may be made without departing from the spirit and scope of the description.

In addition, the logic flows depicted in the figures do not require the particular order shown, or sequential order, to achieve desirable results. In addition, other steps may be provided, or steps may be eliminated, from the described flows, and other components may be added to, or removed from, the described systems. Accordingly, other embodiments are within the scope of the following claims.

1. A computer-implemented method for monitoring a clinical state of a patient during calcium citrate hemofilter therapy to support the selection of required adjustments to the calcium citrate hemofilter therapy, comprising:
receiving, from a data source providing blood gas analysis sensor data, time series of sampled measurement values for the patient's blood gas state parameters comprising systemic calcium, postfilter calcium, pH, bicarbonate, sodium, potassium and chloride;
receiving, from a plurality of calcium citrate hemofilter analysis sensors, time series of continuous measurement values for calcium citrate hemofilter state parameters comprising blood flow, dialysate flow, citrate infusion rate, and calcium infusion rate parameter settings;
mapping each blood gas analysis state parameter to predefined calcium citrate hemofilter adjustment reference tables defining if a particular parameter is within a normal range, or too low, or too high, and what adjustments are appropriate to return all parameters to normal range, and generating one or more therapy adjustment suggestion outputs based on one or more of the blood gas analysis state parameters;
mapping each state parameter to a predefined corresponding graphical representation with each graphical representation for a particular state parameter being distinct from all graphical representations of remaining state parameters;
rendering, in a virtual scene representation of an inside of a blood filled hemofilter catheter representing blood flowing through the hemofilter, animated visualizations of the graphical representations in accordance with predefined animation rules, such that respective graphical objects move through the inside of the catheter and reflect current values of the respective state parameters and the generated one or more therapy adjustment suggestion outputs.

2. The method of claim 1, wherein the predefined animation rules comprise:
for calcium citrate hemofilter settings parameters blood flow and dialysate flow, citrate infusion rate, and calcium infusion rate:
Flow rates of red blood cells, dialysate droplets, calcium ions showing calcium infusion flow and calcium ions showing calcium infusion rate are rendered at a standard animation pace of 8 to 12 mm/s for indicating normal flow rates for the respective parameters for said patient;
Flow rates of red blood cells showing blood flow, dialysate droplets showing dialysate flow, calcium ions showing calcium infusion flow and calcium ions showing calcium infusion rate are rendered at two to four times slower pace than the standard animation pace, for indicating too low flow rates for the respective parameters suggesting a corresponding increase to a user;
Flow rates of red blood cells showing blood flow, dialysate droplets showing dialysate flow, calcium ions infused after the filter showing calcium infusion flow and citrate ions showing citrate infusion rate are rendered at two to four times faster pace than the standard animation pace for indicating too high flow rates for the respective parameters suggesting a corresponding reduction to the user.

3. The method of claim 1, wherein the predefined animation rules comprise:
For calcium citrate hemofilter state parameters citrate infusion, calcium infusion and dialysate flow:
If an infusion rate of citrate, calcium and dialysate is within normal limits and does not require rate changing, rendering an infusion source object having a predefined standard size representing an infusion bag of a solution;
If the infusion rate of citrate, calcium and dialysate is too high and needs to be reduced, rendering the infusion source object with a size that is enlarged by at least factor 1.5 compared to the predefined standard size representing this parameter within a normal range;
If the infusion rate of citrate, calcium and dialysate is too low and needs to be increased, rendering the infusion source object with a size that is reduced by at least factor 1.5 compared to the predefined standard size.

4. The method of claim 1, wherein the predefined animation rules comprise:
For calcium citrate hemofilter state parameters citrate infusion, calcium infusion and dialysate flow:
If an individual infusion rate of citrate or calcium is within normal limits without a need for rate changing, rendering individual citrate and calcium objects with a continuous contour;
If the individual infusion rate of citrate or calcium is too high with a need for rate reduction, rendering individual citrate and calcium objects with a continuous contour;
If the individual infusion rate of citrate or calcium is too low with a need for increase, rendering individual citrate and calcium objects with a dashed-line contour.

5. The method of claim 1, wherein the predefined animation rules comprise:
For the systemic calcium state parameter:
Systemic calcium ions measured in arterial or venous blood gas analysis are rendered as systemic calcium ion objects flowing in the scene towards the filter with substantially a 1:1 ratio to citrate ion objects representing infused citrate ions for indicating normal systemic calcium concentration;
Systemic calcium ion objects flowing in the scene towards the filter are rendered at a calcium/citrate object ratio to the infused citrate ions in the range of 1:2 to 1:5 for indicating too low systemic calcium concentration;
Systemic calcium ion objects flowing in the scene towards the filter are rendered at a calcium/citrate ratio to the infused citrate ions in the range of 2:1 to 5:1 for indicating too high systemic calcium concentration.

6. The method of claim 1, wherein the predefined animation rules comprise:
For the systemic calcium state parameter:
If systemic calcium ions measured in arterial or venous blood gas analysis flowing in the scene towards the filter are within normal limits, rendering individual systemic calcium ion objects with a continuous line contour flowing in a single level of the blood-filled catheter;
If systemic calcium ions measured in arterial or venous blood gas analysis flowing in the scene towards the filter are too high, rendering the individual systemic calcium ion objects with a continuous line contour flowing on multiple levels of the blood-filled catheter;
If systemic calcium ions measured in arterial or venous blood gas analysis flowing in the scene towards the filter are too low, rendering the individual systemic calcium ion objects with a dashed-line contour flowing in a single level of the blood-filled catheter.

7. The method of claim 1, wherein the predefined animation rules comprise:
For a postfilter calcium concentration state parameter:
If a postfilter calcium level is measured as too low in postfilter calcium blood gas analysis, rendering a single blinking calcium ion object;
If the postfilter calcium level is measured as too high in the postfilter calcium blood gas analysis, rendering a cluster of a plurality of multiple blinking calcium ion objects.

8. The method of claim 7, wherein blinking objects have two alternating states comprising a solid object visualization state and a non-solid object state, a blinking object in the non-solid object state being outlined with dashed lines.

9. The method of claim 1, wherein the predefined animation rules comprise:
For metabolic alkalosis or acidosis state parameters:
If a normal pH parameter of a patient—neither metabolic alkalosis nor acidosis state—is measured in arterial blood gas analysis, substantially a 1:1 ratio of hydrogen ion objects to bicarbonate ion objects is rendered;
If the measured pH parameter of the patient indicates metabolic alkalosis state, a ratio in the range of 1:2 to 1:5 of hydrogen ion objects to bicarbonate ion objects is rendered;
If the measured pH parameter of the patient indicates metabolic acidosis state, a ratio in the range of 2:1-5:1 of hydrogen ion objects to bicarbonate ion objects is rendered.

10. The method of claim 1, wherein the predefined animation rules comprise:
For citrate accumulation state parameters:
in case of a normal state with no-citrate accumulation, an anion gap object is rendered with a predefined normal size in comparison to other flowing objects, wherein the anion gap object is present within a plurality of at least two objects in a 20-30 sec long stream visualization and systemic calcium concentration depending on actual measured systemic calcium concentration;
in case of citrate accumulation, the anion gap object is increased in size compared to the predefined normal size.

11. A computer readable medium comprising program instructions that, when loaded into a memory of a computing device and executed by at least one processor of the computing device, cause the at least one processor to execute the following steps for monitoring a clinical state of a patient during calcium citrate hemofilter therapy to support a selection of required adjustments to the calcium citrate hemofilter therapy:
receiving, from a data source providing blood gas analysis sensor data, time series of sampled measurement values for the patient's blood gas state parameters comprising systemic calcium, postfilter calcium, pH, bicarbonate, sodium, potassium and chloride;
receiving, from a plurality of calcium citrate hemofilter analysis sensors, time series of continuous measurement values for calcium citrate hemofilter state parameters;
mapping each blood gas analysis state parameter to predefined calcium citrate hemofilter adjustment reference tables defining if a particular parameter is within a normal range, or too low, or too high, and what adjustments are appropriate to return all parameters to normal range, and generating one or more therapy adjustment suggestion outputs based on one or more of the blood gas analysis state parameters;
mapping each state parameter to a predefined corresponding graphical representation with each graphical representation for a particular state parameter being distinct from all graphical representations of remaining state parameters;
rendering, in a virtual scene representation of an inside of a blood filled hemofilter catheter representing blood flowing through the hemofilter, animated visualizations of the graphical representations in accordance with predefined animation rules, such that respective graphical objects move through the inside of the catheter and reflect current values of the respective state parameters and the generated one or more therapy adjustment suggestion outputs.

12. The computer readable medium of claim 11 comprising further program instructions implementing the following predefined animation rule:
for calcium citrate hemofilter state parameters blood flow and dialysate flow, citrate infusion rate, and calcium infusion rate:
Flow rates of red blood cells, dialysate droplets, calcium ions showing calcium infusion flow and calcium ions showing calcium infusion rate are rendered at a standard animation pace of 8 to 12 mm/s for indicating normal flow rates for the respective parameters for said patient;
Flow rates of red blood cells showing blood flow, dialysate droplets showing dialysate flow, calcium ions showing calcium infusion flow and calcium ions showing calcium infusion rate are rendered at two to four times slower pace than the standard animation pace, for indicating too low flow rates for the respective parameters suggesting a corresponding increase to a user;
Flow rates of red blood cells showing blood flow, dialysate droplets showing dialysate flow, calcium ions infused after the filter showing calcium infusion flow and citrate ions showing citrate infusion rate are rendered at two to four times faster pace than the standard animation pace for indicating too high flow rates for the respective parameters suggesting a corresponding reduction to the user.

13. The computer readable medium of claim 11 comprising further program instructions implementing the following predefined animation rule:
For calcium citrate hemofilter state parameters citrate infusion, calcium infusion and dialysate flow:
If an infusion rate of citrate, calcium and dialysate is within normal limits and does not require rate changing, rendering an infusion source object having a predefined standard size representing an infusion bag of a solution;
If the infusion rate of citrate, calcium and dialysate is too high and needs to be reduced, rendering the infusion source object with a size that is enlarged by at least factor 1.5 compared to the predefined standard size representing this parameter within a normal range;
If the infusion rate of citrate, calcium and dialysate is too low and needs to be increased, rendering the infusion source object with a size that is reduced by at least factor 1.5 compared to the predefined standard size.

14. The computer readable medium of claim 11 comprising further program instructions implementing the following predefined animation rule:
For calcium citrate hemofilter state parameters citrate infusion, calcium infusion and dialysate flow:
If an individual infusion rate of citrate or calcium is within normal limits without a need for rate changing, rendering individual citrate and calcium objects with a continuous contour;
If the individual infusion rate of citrate or calcium is too high with a need for rate reduction, rendering individual citrate and calcium objects with a continuous contour;
If the individual infusion rate of citrate or calcium is too low with a need for increase, rendering individual citrate and calcium objects with a dashed-line contour.

15. The computer readable medium of claim 11 comprising further program instructions implementing the following predefined animation rule:
For the systemic calcium state parameter:
Systemic calcium ions measured in arterial or venous blood gas analysis are rendered as systemic calcium ion objects flowing in the scene towards the filter with substantially a 1:1 ratio to citrate ion objects representing infused citrate ions for indicating normal systemic calcium concentration;
Systemic calcium ion objects flowing in the scene towards the filter are rendered at a calcium/citrate object ratio to the infused citrate ions in the range of 1:2 to 1:5 for indicating too low systemic calcium concentration;
Systemic calcium ion objects flowing in the scene towards the filter are rendered at a calcium/citrate ratio to the infused citrate ions in the range of 2:1 to 5:1 for indicating too high systemic calcium concentration.

16. The computer readable medium of claim 11 comprising further program instructions implementing the following predefined animation rule:
For the systemic calcium state parameter:
If systemic calcium ions measured in arterial or venous blood gas analysis flowing in the scene towards the filter are within normal limits, rendering individual systemic calcium ion objects with a continuous line contour flowing in a single level of the blood-filled catheter;
If systemic calcium ions measured in arterial or venous blood gas analysis flowing in the scene towards the filter are too high, rendering the individual systemic calcium ion objects with a continuous line contour flowing on multiple levels of the blood-filled catheter;
If systemic calcium ions measured in arterial or venous blood gas analysis flowing in the scene towards the filter are too low, rendering the individual systemic calcium ion objects with a dashed-line contour flowing in a single level of the blood-filled catheter.

17. The computer readable medium of claim 11 comprising further program instructions implementing the following predefined animation rule:
For a postfilter calcium concentration state parameter:
If a postfilter calcium level is measured as too low in postfilter calcium blood gas analysis, rendering a single blinking calcium ion object;
If the postfilter calcium level is measured as too high in the postfilter calcium blood gas analysis, rendering a cluster of a plurality of multiple blinking calcium ion objects.

18. The computer readable medium of claim 11 comprising further program instructions implementing the following predefined animation rule:
For metabolic alkalosis or acidosis state parameters:
If a normal pH parameter of a patient—neither metabolic alkalosis nor acidosis state—is measured in arterial blood gas analysis, a ratio of hydrogen ion objects to bicarbonate ion objects of substantially 1:1 is rendered;
If the measured pH parameter of the patient indicates metabolic alkalosis state, a ratio in the range of 1:2 to 1:5 of hydrogen ion objects to bicarbonate ion objects is rendered;
If the measured pH parameter of the patient indicates metabolic acidosis state, a ratio in the range of 2:1-5:1 of hydrogen ion objects to bicarbonate ion objects is rendered.

19. The computer readable medium of claim 11 comprising further program instructions implementing the following predefined animation rule:
For citrate accumulation state parameters:
in case of a normal state with no-citrate accumulation, an anion gap object is rendered with a predefined normal size in comparison to other flowing objects, wherein the anion gap object is present within a plurality of at least two objects in a 20-30 sec long stream visualization and systemic calcium concentration depending on actual measured systemic calcium concentration;
in case of citrate accumulation, the anion gap object is increased in size compared to the predefined normal size.

20. A computer system for monitoring a clinical state of a patient during calcium citrate hemofilter therapy to support a selection of required adjustments to the calcium citrate hemofilter therapy, comprising:
an interface adapted to receive time series of sampled measurement values obtained from a plurality of blood gas analysis sensors for the patient's blood gas state parameters comprising: systemic calcium concentration from arterial or venous blood sample, postfilter calcium concentration from postfilter blood gas analysis, pH and bicarbonate ions from arterial blood gas analysis, and further adapted to receive time series of sampled measurement values obtained from a plurality of calcium citrate hemofilter analysis sensors for the following calcium citrate hemofilter state parameter settings comprising: blood flow, dialysate flow, citrate infusion rate, and calcium infusion rate parameter settings;
an integrator module adapted to integrate received blood gas analysis data and current calcium citrate hemofilter settings with calcium citrate hemofilter therapy adjustment reference tables to generate suggestions for therapy adjustment;
a mapper module adapted to map each blood gas state parameter and therapy adjustment data output to a predefined corresponding graphical representation with each graphical representation for a particular state parameter or particular therapy adjustment data output being distinct from all graphical representations of remaining state parameters and therapy adjustment data outputs; and
a renderer module adapted to render, in a virtual blood-filled catheter inside scene representing an inside of a hemofilter catheter, animated visualizations of the graphical representations, in accordance with predefined animation rules, such that respective graphical objects move through the inside of the catheter and reflect current values of the respective state parameters and therapy adjustment data outputs.
